SEQUENCE LISTING (29 sequences)

SEQ ID NO: 1 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 2 - DNA, 1503 bp, Artificial Sequence
  SERPING1 wild type sequence
atggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctga

SEQ ID NO: 3 - DNA, 1503 bp, Homo sapiens
atggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctag

SEQ ID NO: 4 - DNA, 1503 bp, Artificial sequence
  Codon-optimized SERPING1 HA03
atggccagcaggctgaccctgctgaccctgctgctgctgctgctggctggagacagggccagcagcaaccccaatgccaccagcagcagcagccaggaccctgagagcctgcaggacaggggagagggcaaggtggctaccacagtgatcagcaagatgctgtttgtggagcccatcctggaggtgagcagcctgcccaccaccaacagcaccaccaacagtgccaccaagatcacagccaacaccacagatgagcccaccacccagcccaccacagagcccaccacccagcccaccatccagcccacccagcccaccacccagctgcccacagacagccccacccagcccaccacaggcagcttctgccctggccctgtgaccctgtgcagtgacctggagagccacagcacagaggctgtgctgggagatgccctggtggacttcagcctgaagctgtaccatgccttcagtgccatgaagaaggtggagaccaacatggccttcagccccttcagcattgccagcctgctgacccaggtgctgctgggagctggagagaacaccaagaccaacctggagagcatcctgagctaccccaaggacttcacctgtgtgcaccaggccctgaagggcttcaccaccaagggagtgaccagtgtgagccagatcttccacagccctgacctggccatcagggacacctttgtgaatgccagcaggaccctgtacagcagcagccccagggtgctgagcaacaacagtgatgccaacctggagctgatcaacacctgggtggccaagaacaccaacaacaagatcagcaggctgctggacagcctgcccagtgacaccaggctggtgctgctgaatgccatctacctgagtgccaagtggaagaccacctttgaccccaagaagaccaggatggagcccttccacttcaagaacagtgtgatcaaggtgcccatgatgaacagcaagaagtaccctgtggcccacttcattgaccagaccctgaaggccaaggtgggccagcttcagcttagccacaacctgagcctggtgatcctggtgccccagaacctgaagcacaggctggaggacatggagcaggccctgagccccagtgtgttcaaggccatcatggagaagctggagatgagcaagttccagcccaccctgctgaccctgcccaggatcaaggtgaccaccagccaggacatgctgagcatcatggagaagctggagttctttgacttcagctatgacctgaacctgtgtggcctgacagaggaccctgacctgcaagtcagtgccatgcagcaccagacagtgctggagctgacagagacaggagtggaggctgctgctgccagtgccatcagtgtggccaggaccctgctggtgtttgaggtgcagcagcccttcctgtttgtgctgtgggaccagcagcacaagttccctgtgttcatgggcagggtgtatgaccccagggcctag

SEQ ID NO: 5 - DNA, 1503 bp, Artificial sequence
  Codon-optimized SERPING1 HA04
atggcctccaggctgaccctgctgaccctcctgctgctgctgctggctggggatagagcctcctcaaatccaaatgctaccagctccagctcccaggatccagagagtttgcaagacagaggagaaggcaaggtggcaacaacagttatctccaagatgctatttgttgaacccatcctggaggtttccagcttgcctacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactggctccttctgcccaggacctgttactctctgctctgacttggagagtcattcaacagaggctgtgttgggagatgctttggtagatttctccctgaagctctaccatgccttctcagcaatgaagaaggtggagaccaacatggccttttccccattcagcattgccagcctccttacccaggtcctgctgggggctggagagaacaccaaaacaaacctggagagcatcctctcttaccccaaggacttcacctgtgtccatcaggccctgaagggcttcactaccaaaggtgtcacctcagtctctcagatcttccatagcccagacctggccataagggacacctttgtgaatgcctctaggaccctgtacagcagcagccccagagtcctaagcaacaacagtgatgccaacttggagctcatcaacacctgggtggccaagaacaccaacaacaagatcagcaggctgctagacagtctgccctctgataccaggcttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaggccaaagtgggccagctccagctgtcccacaatctgagtttggtgatcctggtaccccagaacctgaaacatagacttgaagacatggaacaggctctcagcccttctgttttcaaggccatcatggagaaactggagatgtccaagttccagcccactctcctaacactacccaggatcaaagtgactaccagccaggatatgctctcaatcatggagaaattggaattctttgatttttcttatgaccttaacctgtgtggcctgacagaggacccagatcttcaggtttctgctatgcagcatcagacagtgctggaactgacagagactggagtggaggctgctgcagcctcagccatctctgtggccaggaccctgctggtctttgaagtgcagcagcccttcctctttgtgctctgggaccagcagcacaagttccctgtcttcatgggcagagtatatgaccccagggcctag

SEQ ID NO: 6 - DNA, 1503 bp, Artificial sequence
  Codon-optimized SERPING1 HA05
atggcctccaggctgaccctgctgaccctcctgctgctgctgctggctggggacagagcctcctcaaatccaaatgctaccagctccagctcccaggacccagagagcctccaagacagaggagaaggcaaggtggcaacaacagtcatctccaagatgctctttgtggaacccatcctggaggtctccagcctccctacaaccaactcaacaaccaattcagccaccaagataacagctaataccactgatgaacccaccacacaacccaccacagagcccaccacccaacccaccatccaacccacccaaccaactacccagctcccaacagactctcctacccagcccactactggctccttctgcccaggacctgtcactctctgctctgacctggagagccactcaacagaggctgtgctgggagatgctctggtggacttctccctgaagctctaccatgccttctcagcaatgaagaaagtggagaccaacatggccttttccccattcagcattgccagcctcctgacccaggtcctgctgggggctggagagaacaccaagacaaacctggagagcatcctctcttaccccaaggacttcacctgtgtccatcaggccctgaagggcttcactaccaagggtgtcacctcagtctctcagatcttccacagcccagacctggccataagggacacctttgtgaatgcctctaggaccctgtacagcagcagccccagagtcctcagcaacaacagtgatgccaacctggagctcatcaacacctgggtggccaagaacaccaacaacaagatcagcaggctgctggacagcctgccctctgacaccaggctggtcctcctcaatgctatctacctgagtgccaagtggaagacaacatttgatcccaagaaaaccagaatggaaccctttcacttcaagaactcagtcataaaggtgcccatgatgaatagcaagaaataccctgtggcccacttcattgaccaaactctcaaggccaaggtgggccagctccagctgtcccacaatctgagcctggtgatcctggtgccccagaacctgaagcacagactggaagacatggaacaggctctcagcccttctgtcttcaaggccatcatggagaagctggagatgtccaagttccagcccactctcctcacactccccaggatcaaggtcactaccagccaggacatgctctcaatcatggagaagctggaattctttgacttttcttatgacctgaacctgtgtggcctgacagaggacccagatctccaggtctctgctatgcagcaccagacagtgctggaactgacagagactggagtggaggctgctgcagcctcagccatctctgtggccaggaccctgctggtctttgaagtgcagcagcccttcctctttgtgctctgggaccagcagcacaagttccctgtcttcatgggcagagtgtatgaccccagggcctag

SEQ ID NO: 7 - DNA, 1503 bp, Artificial Sequence
  Codon-optimized SERPING1 HA06
atggcctccaggctgaccctgctgaccctcctgctgctgctgctggctggggacagggcctcctcaaaccccaatgccaccagctccagctcccaggacccagagagcctccaggacaggggggaaggcaaggtggctaccacagtcatctccaagatgctctttgtggagcccatcctggaggtctccagcctccccaccaccaactcaaccaccaactcagccaccaagatcacagccaacaccactgatgagcccaccacacagcccaccacagagcccaccacccagcccaccatccagcccacccagcccaccacccagctccccacagactcccccacccagcccaccactggctccttctgcccaggccctgtcaccctctgctctgacctggagagccactcaacagaggctgtgctgggggatgccctggtggacttctccctgaagctctaccatgccttctcagccatgaagaaggtggagaccaacatggccttctcccccttcagcattgccagcctcctgacccaggtcctgctgggggctggggagaacaccaagaccaacctggagagcatcctctcctaccccaaggacttcacctgtgtccatcaggccctgaagggcttcaccaccaagggagtcacctcagtctcccagatcttccacagcccagacctggccatcagggacacctttgtgaatgcctccaggaccctgtacagcagcagccccagggtcctcagcaacaacagtgatgccaacctggagctcatcaacacctgggtggccaagaacaccaacaacaagatcagcaggctgctggacagcctgccctctgacaccaggctggtcctcctcaatgccatctacctgagtgccaagtggaagaccacctttgatcccaagaagaccaggatggaacccttccacttcaagaactcagtcatcaaggtgcccatgatgaacagcaagaagtaccctgtggcccacttcattgaccagaccctcaaggccaaggtgggccagctccagctgtcccacaacctgagcctggtgatcctggtgccccagaacctgaagcacaggctggaggacatggaacaggccctcagcccctctgtcttcaaggccatcatggagaagctggagatgtccaagttccagcccaccctcctcaccctccccaggatcaaggtgaccaccagccaggacatgctctcaatcatggagaagctggagttctttgacttctcctatgacctgaacctgtgtggcctgacagaggacccagatctccaggtctctgccatgcagcaccagacagtgctggaactgacagagactggagtggaggctgctgcagcctcagccatctctgtggccaggaccctgctggtctttgaagtgcagcagcccttcctctttgtgctctgggaccagcagcacaagttccctgtcttcatgggcagggtgtatgaccccagggcctag

SEQ ID NO: 8 - DNA, 1503 bp, Artificial Sequence
  Codon-optimized SERPING1 HA11
atggcatcaaggctgacccttctcaccctgctcctgctgcttctggctggggacagagcctccagcaaccccaatgccacctccagcagctctcaagatccagagtccctgcaagaccggggagaaggcaaggtggctaccacagtcatctccaagatgctctttgtggaacccatcctggaagtgtcctccctccccaccactaactccaccacaaactctgccactaagatcacagccaacactacagatgagccaactacccagcctacaacagagcctaccacccaaccaaccatccagccaacccagcccaccactcagctgcccacagacagcccaacacagcccacaactggctccttctgccctggccctgtgaccctctgcagtgacttggaaagccacagcaccgaggctgtgctgggagatgctctggtggacttttctctcaagctgtaccatgctttctcagcaatgaagaaagtagagaccaacatggccttcagccccttttccattgccagcctcctcactcaagtgctcctgggggctggagagaataccaaaaccaacctggagagcatcctctcctaccccaaggacttcacctgtgtgcaccaagccctgaaaggcttcacaaccaagggggtgacttctgtctcccagatcttccactctccagacctggccatcagagacaccttcgtgaatgcaagtaggaccctctacagctcctccccaagggtgctgtccaacaattctgatgccaatctggagctcatcaacacctgggtggccaagaacacaaacaacaagatttctaggctcctggactccctgccttctgacacaagactggttctgctgaatgctatctacctgtctgcaaagtggaagaccacctttgaccccaaaaagacaaggatggagcccttccattttaagaattcagtgatcaaggtgcccatgatgaacagcaagaagtaccctgttgcccacttcattgaccaaacactgaaggccaaagtgggccagctccagctttcccacaacctcagcctggtgattctggtcccccagaacttgaagcaccggctggaggacatggagcaagcactgagccccagtgtcttcaaggcaatcatggaaaaactggagatgtccaaattccagcccactctgttgaccttgcccagaatcaaagtcaccaccagccaagatatgctgagtatcatggagaagcttgagttctttgatttctcctatgatctgaacctgtgtggcctgacagaagaccctgacctccaagtcagtgccatgcagcaccagacagtgttggagctgactgagactggtgtggaggcagcagccgcctcagccatctctgtggccagaaccctgctggtgtttgaggtgcagcagcctttcctgtttgtcctgtgggaccagcaacacaagttcccagtgttcatgggcagagtgtatgacccaagggcctga

SEQ ID NO: 9 - DNA, 1503 bp, Artificial Sequence
  Codon-optimized SERPING1 HA12
atggcaagcagactgaccttgctgacactgctccttctcctgctggctggagaccgggccagcagcaaccccaatgccacttcttcctccagtcaagacccagagtccctgcaagacagaggagaaggcaaggttgctaccactgtgatctccaagatgctgttcgtggagcccatcctggaagtgtccagcctgcccaccactaactccaccacaaacagtgccaccaaaatcacagccaacactacagatgagccaaccacccaaccaaccacagaacccacaacacagcctaccatccagcctactcagcccaccacccagcttcctacagactcccccacccagccaacaactggctccttctgccctggccctgtgactctgtgctctgacctggagagccacagcaccgaggctgtgctgggagatgctctggtggacttctccctgaaactgtaccacgccttttcagccatgaagaaagtagagaccaacatggccttcagcccctttagcattgcctccctcctcactcaagttctgctgggggctggggagaacacaaagaccaacctggaaagcatcctctcctaccccaaggacttcacctgtgtgcaccaagcactcaagggcttcacaaccaagggggtgaccagtgtctcccagatcttccattccccagacctggccatcagagacacatttgtgaatgcatccagaaccctctacagctcctctccaagggtgctgtccaacaactcagatgccaatcttgagctgatcaacacctgggtggccaaaaataccaataacaagatctcaaggctgctggacagcctccccagtgacaccagacttgtgctcctgaatgctatctacctgtctgcaaagtggaagactacctttgatcccaaaaagacaaggatggagcctttccacttcaagaactctgtcatcaaagtgcccatgatgaacagcaagaagtaccctgttgcccactttattgatcagaccctgaaggccaaggtgggccagctccagctgtcccacaacctttctctggtgattctggtcccccagaacctgaagcacagattggaagacatggagcaagccctgagcccttcagtgtttaaggcaatcatggagaagctggagatgtccaaattccagcccactctcctcaccctgcctaggatcaaggtcaccaccagccaagatatgctctctatcatggaaaagttggagttctttgacttttcctatgatctgaacctctgtggcctgactgaggaccctgacctccaagtgtcagcaatgcagcaccagacagtgctggagctcacagagactggggtggaagccgcagcagcctctgccatctctgtggctaggacccttctggtctttgaggtgcagcagcccttcctgtttgtcctgtgggaccagcaacacaagttcccagtgttcatgggcagagtgtatgaccccagagcctga

SEQ ID NO: 10 - DNA, 1503 bp, Artificial Sequence
  Codon-optimized SERPING1 HA13
atggcatctaggctgaccttgctcactctgctgcttctcctcctggctggagaccgggcctccagcaaccccaatgctacctcctccagttcccaagatccagagtccctgcaagacagaggagaaggcaaggtggcaaccactgtcatcagcaagatgctgtttgtggaacccatcctggaagtgtccagcctccccacaaccaactccaccacaaattcagccaccaagatcactgccaacacaactgatgagcccaccacccaaccaaccacagagccaactacccagcccaccatccagcccactcagcctaccacccagcttcctactgacagcccaacacagccaaccactggctccttctgccctggccctgtgaccctgtgctctgacttggaaagccacagcactgaagctgtgctgggagatgctctggtggacttttccctcaagctgtaccacgccttctctgcaatgaagaaggttgagaccaacatggccttttctcccttcagcattgccagcctgctgactcaagtgctgctgggggctggggagaacaccaaaacaaacctggagagcatcctctcctaccccaaagatttcacctgtgtgcaccaagccctgaaaggcttcacaacaaagggggtcacctctgtctcccagatcttccattcccctgacctggccatcagagacaccttcgtgaatgccagcagaaccctctacagcagctccccaagggtgctctccaacaattctgatgccaatctggagctcatcaacacctgggtggccaaaaacactaacaacaagatttccagactcctggactccctgcccagtgacacaaggttggtcctcctgaatgcaatctacctgtctgctaagtggaagaccacctttgatcccaagaaaaccagaatggaaccattccactttaagaactcagtgatcaaggtccccatgatgaacagcaaaaagtaccctgttgcccacttcattgatcagaccctgaaggccaaggtgggccagctccagctgtcccacaacctctctctggtcattctggtgccccagaacttgaagcaccggctggaagatatggagcaagcactgagcccctctgtgttcaaggcaattatggagaagcttgagatgtccaagttccagcctacactgctcaccctgcctaggatcaaagtgactaccagccaagacatgctctcaatcatggagaaactggagttctttgacttctcctatgatctgaacctgtgtggcctgactgaggacccagacctccaagtcagtgccatgcagcaccagacagtgttggagctgacagaaactggggtggaggcagcagccgcctctgccatcagtgtggctaggaccctgttggtgtttgaggtgcagcagccctttctctttgtcctgtgggaccagcaacacaagtttcctgtcttcatgggcagagtgtatgaccccagagcctga

SEQ ID NO: 11 - DNA, 1503 bp, Artificial Sequence
  Codon-optimized SERPING1 HA14
atggccagcagactcaccctcctcactctgctcctgcttctgctggctggggaccgggcctctagcaaccccaatgccaccagctcctccagccaagatccagaatctctgcaagacagaggagaaggcaaggttgccactactgtgatcagcaagatgctctttgtggagcccatcctggaagtgagcagcctccccactaccaacagcaccactaactcagctaccaagatcacagccaataccacagatgaacccaccacacagcctacaacagaacctaccactcaacccaccatccagcctactcagcccaccacccagctgcccacagactccccaacccagccaacaactggctccttctgccctggccctgtcacactctgcagtgacttggagagccacagcactgaggctgtgctgggagatgccctggtggacttttccctcaagctctaccacgccttctcagcaatgaagaaagtagagaccaacatggcattctctcctttcagcattgcctccctgctgacccaagtcctcctgggggctggagagaacactaaaaccaatcttgagtccatcctctcctaccccaaggacttcacctgtgtccaccaagcactgaaaggcttcacaaccaaaggagtgacctctgtgtcccagatcttccattcccctgatctggccatcagagacaccttcgtgaatgcttccagaaccttgtacagctctagcccaagggtgctgtccaacaattctgatgctaatctggagctgatcaacacctgggtggccaaaaacacaaacaacaagatttctaggctcctggacagcctgccttctgacacaaggctggttctgctgaatgcaatctacctgtctgcaaagtggaagaccacctttgaccccaaaaagacaaggatggagccattccacttcaagaacagtgtcatcaaagtgcccatgatgaactccaagaagtacccagtggcccatttcattgatcagaccctgaaggccaaggtgggccagctccagctttcccacaacctctcactggtgattctggtcccccagaacctgaagcaccggctggaagatatggagcaagccctcagccccagtgtgtttaaggctatcatggagaagctggagatgtccaaattccagcccactctcctgacactgccaaggatcaaggtcaccacctcccaagacatgctgtcaatcatggaaaaactggagttctttgatttctcctatgacctgaacctgtgtggcctgactgaggaccctgacctccaagtgtctgccatgcagcaccagacagtgctggagctcacagagactggggtggaagccgctgctgcctcagccatctcagtggctaggaccctgctggtctttgaggtgcagcagcccttcctgtttgtcctgtgggaccagcaacacaagttccctgtgttcatgggcagagtgtatgatcccagagcctga

SEQ ID NO: 12 - DNA, 1503 bp, Artificial Sequence
  Codon-optimized SERPING1 HA15
atggcatctaggctgacactgctcaccttgctcctgctgcttctggctggggacagagcttcctccaaccccaacgccacctccagctcttcccaagaccctgagtctctgcaagataggggggagggcaaggtggctaccacagtcatcagcaagatgctctttgtggaacccatcctggaagtgagcagcctccccactaccaatagcaccaccaacagtgccacaaagatcacagccaacactacagatgagccaaccactcagcccacaacagagcctactacccagcccaccatccagcccacacagcctaccacccagcttcctacagacagccccactcaacccaccactggctccttctgccctggccctgtgaccctctgcagtgacttggagagccacagcactgaggctgtgctgggagatgctctggtggacttttccctcaagctctaccacgccttctctgcaatgaagaaggttgagaccaacatggcctttagcccattcagcattgcctccttgctgacccaagtcctgctgggggctggagagaacaccaaaacaaatcttgagtccatcctctcctaccccaaagacttcacctgtgtgcaccaagcactgaaaggcttcacaaccaagggggtgacttctgtgtcccagatcttccactctcctgacctggccatcagagacacctttgtcaatgccagcagaaccctttacagctcctccccaagagtcctcagcaacaattctgatgccaatctggagctgatcaacacctgggtggcaaaaaacacaaacaacaagatttccagactcctggactccctgccttctgacacaaggctggttctgctgaatgctatctacctgtctgccaagtggaagaccacatttgaccccaagaaaaccagaatggagcccttccatttcaagaactcagtgatcaaggtccccatgatgaacagcaaaaagtaccctgttgcccacttcattgatcagaccctgaaggccaaagtgggccagctccagctgtcccacaacctgagcctggtcattctggtgccccagaacctgaagcacagactggaagatatggagcaagccctgagcccttcagtctttaaggcaatcatggagaagctggagatgtccaagttccagccaactctcctcactctgcccagaatcaaagtcaccaccagccaagacatgctgagtatcatggaaaaactggagttctttgatttctcctatgacctgaacctctgtggcctgactgaggacccagacctccaagtgtctgccatgcagcaccagacagtgctggagctcacagaaactggtgtggaggccgctgctgcctcagccatcagtgtggcccgcaccctgttggtgtttgaggtgcagcagcctttcctgttcgtgctgtgggaccagcaacacaagtttccagtgttcatgggcagagtgtatgatccaagggcctga

SEQ ID NO: 13 - DNA, 1503 bp, Artificial Sequence
  Codon-optimized SERPING1 HA16
atggcctctaggctcaccctgctgacactgctgctcctcctgctggctggagaccgggccagcagcaaccccaatgctaccagctcctccagtcaagaccctgagtccctgcaagacagaggagaaggcaaggttgccactacagtcatctccaagatgctgtttgtagagcccatcctggaagtgtccagcctgcctaccaccaatagcaccaccaacagtgccaccaagatcacagccaataccacagatgagccaaccacccagcctacaacagagcctaccactcaacccaccatccagccaacacagcctactacccagctgcccacagacagcccaactcagcccaccactggctccttctgccctggccctgtcacactctgcagtgacctggaaagccacagcactgaggctgtgctgggagatgctctggtggacttttctctgaaactgtaccacgccttctctgcaatgaagaaagtggagaccaacatggcattctccccattcagcattgcctccttgctgacccaagtgctcctgggggctggggagaacacaaaaacaaacctggagagcatcctctcctaccccaaggacttcacctgtgtgcaccaagccctcaagggcttcacaaccaaaggagtgacctctgtgagccagatcttccattcccctgacctggccattagggacaccttcgtgaatgcaagccggaccctctacagcagctctccaagagtcctgtccaacaattctgatgccaacctagagctgatcaacacctgggtggcaaagaacactaacaacaagatttccagactcctggactccctcccatctgacacaaggctggtcctcctgaatgccatctacctgtctgctaagtggaagaccacatttgaccccaaaaagacaaggatggaacccttccacttcaaaaactcagtgatcaaagtccccatgatgaacagcaagaagtaccctgtggcccatttcattgatcagaccctgaaggccaaggtgggccagctccagctttcccacaacctcagccttgtgattctggtgccccagaacttgaagcacagactggaggacatggagcaagccctgagccccagtgtcttcaaggcaatcatggagaaacttgagatgtccaaattccagcccactctgctcactctccccagaatcaaggtcaccacctcccaagatatgctctcaattatggagaagctggagttctttgatttttcctatgatctgaacctgtgtggcctgactgaagatccagacctccaagtctctgccatgcagcaccagacagtgttggagctcacagaaactggggtggaagccgctgctgcctcagccatcagtgtggctaggaccctgttggtgtttgaggtgcagcagccctttctctttgtgctgtgggaccagcaacacaagttcccagtgttcatgggcagagtgtatgacccaagggcctga

SEQ ID NO: 14 - DNA, 1503 bp, Artificial Sequence
  Codon-optimized SERPING1 HA17
atggcatcccgcctgaccctcctcaccctgcttctgctcctgctggctggagatagggcctccagcaaccccaatgcaaccagcagctccagccaagaccctgagtccctgcaagacagaggagaaggcaaggttgccaccactgtgatttccaagatgctgtttgtggaacccatcctggaagtgagcagcctcccaacaaccaacagcaccaccaatagtgccacaaaaattacagccaataccacagatgagcctaccactcagcctacaacagaacccaccacccagcctaccatccagccgacccagccaaccacacagctccccactgactcccccacacagcccaccactggctccttctgccctggacctgtgactctgtgctctgacctggaatctcactccactgaggctgtgctgggggatgctctggtggatttttccctcaagctctaccatgccttctctgccatgaagaaggtggagaccaacatggccttttctcccttttccattgccagcctcctgacccaagtgctcctgggagctggggagaacaccaagaccaaccttgagagcattctgtcctaccccaaggactttacctgtgtgcaccaagccctgaaaggcttcaccaccaaaggggtgacctctgtctcccagatcttccacagccctgatctggccattagggacacctttgtcaatgcttccagaactctctattcttcctccccacgggtcctctccaacaattcagatgccaacctggagctgatcaacacctgggtggccaaaaacacaaacaacaagatctcaaggctcctggacagcctccccagtgacacacggctggtgctcctgaatgccatctacctgtctgccaagtggaagaccacttttgacccaaaaaagacaaggatggagcccttccatttcaagaacagtgtgatcaaggtccccatgatgaatagcaagaaataccctgttgcccacttcattgatcagaccctgaaggccaaagtgggccagctgcagttgtcccacaacctcagcctggtcatactggtgccccagaacctgaagcataggctggaagacatggaacaagcactgagcccctcagtgttcaaagctatcatggagaagctggagatgtccaaattccagcccaccctgttgaccctgcctaggatcaaagtcaccacctcccaagatatgctctcaataatggagaaactggagttctttgatttctcctatgatctcaatctgtgtggcctgactgaagatccagacctccaagtctcagcaatgcagcaccagacagtcctggagctcacagagactggtgtggaggcggcagcagcctcagccatctctgtggccagaaccctgctggtgtttgaggtgcagcagcccttcctgtttgttctgtgggaccagcagcataagtttcctgtcttcatgggtagggtgtatgaccccagagcctga

SEQ ID NO: 15 - DNA, 1503 bp, Artificial Sequence
  Codon-optimized SERPING1 HA18
atggccagcagactcaccctgctgaccctgctcctcctgctcctggctggggacagagcctcctccaaccccaatgcaacctcctccagctcccaagaccctgagtccctgcaagataggggagaaggcaaggttgccactactgtcataagcaagatgctgtttgttgaacccatcttggaggtgtccagtctccccaccacaaacagcaccaccaactctgccaccaagatcactgccaataccacagatgaacccaccactcagcccacaactgagcccaccacccagccaaccatacagcctacacagccaacaacccagctgcccactgactccccaacccagcctaccactggcagcttctgccctggtcctgtcaccctctgcagtgacctggaaagccatagcacagaggctgtgctgggggatgctctggtggacttctccctcaaactctaccatgctttttctgcaatgaagaaggtggaaaccaacatggccttcagccccttctccattgccagcctcttgacccaagtgctcctgggagctggagagaacactaagactaatctggagagcatcctctcctaccccaaagacttcacctgtgtgcaccaagccctgaagggcttcaccaccaaaggagtgacctctgtctcccagatcttccattcccctgacctggccataagggacacctttgtgaatgcctctaggaccctgtactccagctctcctcgggtcctcagcaacaactcagatgccaacctggagctcatcaacacctgggtggccaaaaataccaataacaagatttcccgcctcctggattccctgccctctgacacacgcctggtgctcctgaatgccatctacctgagtgccaagtggaagaccacctttgatcccaagaaaaccagaatggagcctttccactttaagaattcagtcattaaagtgcccatgatgaattccaagaagtatcctgtggctcacttcattgaccagaccctcaaggccaaggtaggtcagctccagctgagccacaacctcagcctggtgatcctggtcccccagaacctgaaacataggctggaggacatggaacaagcactgtccccatcagtcttcaaggctatcatggagaagctggagatgagcaaattccagcccacactgctgaccttgcctaggatcaaggtcaccaccagccaagatatgctctcaattatggagaaactggaattctttgacttttcctatgacctcaatctgtgtggcctgacagaagatccagatctccaagtcagtgccatgcagcaccagacagtgctggagctgactgaaactggggtggaggcagcggcagcctcagccatttctgtggcaaggaccctgctggtgtttgaagtgcagcagccctttctctttgtcctgtgggatcagcagcacaagtttccagtgttcatgggccgggtgtatgatccaagggcctga

SEQ ID NO: 16 - DNA, 1503 bp, Artificial Sequence
  Codon-optimized SERPING1 HA19
atggcctcccggctgacccttctcaccctgctcctgctgcttctggctggggaccgggccagcagcaaccccaatgctaccagctcttcatcacaagaccctgagagcctgcaagacagaggagaaggcaaggttgccactactgtgatcagcaagatgctcttcgtggagcccatcctggaagtgtcttctctgcccacaaccaactccaccacaaattctgccaccaaaatcacagccaataccactgatgaacccaccactcaacccaccacagagccaacaacacagccaaccatccagcctacccagcctaccacccagcttcctacagacagcccaactcagcccacaactggctccttctgccctgggcctgtcaccctgtgctctgacttggaaagccacagcaccgaggctgtgctgggagatgctctggtggacttttctttgaagctctaccacgccttcagtgccatgaagaaagtagagaccaacatggcattcagccccttttccattgcttccctgttgacccaagtgctccttggagctggagagaacactaaaaccaatcttgagtccatcctctcctaccccaaagacttcacctgtgtgcaccaagcactgaaaggcttcacaaccaagggggtgacctcagtgtcccagatcttccattccccagacctggccattagggacacatttgtgaatgcaagcagaactctgtacagctccagccctagggtgctgtccaacaattcagatgccaacttggagctcatcaacacctgggtggccaaaaacacaaacaacaagatctccagactcctggactccctccccagtgacaccagactggttctgctgaatgccatctacctgtcagccaagtggaagaccacctttgaccccaagaagacaaggatggaaccattccactttaagaacagtgtcatcaaagtgcccatgatgaacagcaaaaagtaccctgtggcccacttcattgatcagaccctcaaggcaaaggtgggccagctgcagctctcccacaacctcagccttgtgattctggtcccccagaacctgaagcacagactggaggacatggaacaagccctgtctccttcagtcttcaaggccatcatggagaagctggagatgtccaagttccagcccactctcctcacactgcctagaatcaaggtcactacctctcaagatatgctgagcatcatggagaaactggagttctttgatttctcctatgacctcaacctgtgtggcctgacagaagatccagatttgcaagtttctgcaatgcagcaccagacagtgctagagctgactgagactggtgtggaagctgctgcagcctctgctatctctgtggcaagaaccttgctggtgtttgaggtgcagcagcccttcctgtttgtcctgtgggaccagcaacacaagtttccagtgttcatgggcagagtgtatgatcccagagcctga

SEQ ID NO: 17 - DNA, 1503 bp, Artificial Sequence
  Codon-optimized SERPING1 HA20
atggcatccagactcacactgctcactctgctgcttctgttgctggctggagaccgggcctccagcaaccccaacgccacttcttcctccagtcaagatcctgagagtctccaagacagaggagaaggcaaggttgctaccactgtgatttccaagatgctctttgtggagcccatcttggaagtgagctccctgcctaccaccaattccaccacaaattcagccaccaaaatcaccgccaacactacagatgagccaaccacccagcccacgacagagcctacaactcagcccaccatccagcctactcaaccaacaacacagctccccacagacagccccacccagccaactactggctccttctgccctgggcccgtgaccctctgcagtgaccttgagagccacagcaccgaggctgtgctgggagatgctctggtggacttttctctgaagctgtaccatgctttctctgcaatgaagaaagtagagaccaacatggccttcagtcccttttccattgccagcctgctgacccaagtgctcctgggcgctggggagaacacaaaaaccaacctggagtccatcctctcctaccccaaggatttcacctgtgtgcaccaagccctgaagggcttcacaaccaagggggtcacctctgtgtcccagatcttccattccccagacctggccatcagagacaccttcgtgaatgcttcccgcaccctctacagcagctctcctcgagtgctatccaacaattctgatgccaatctggagctcatcaacacctgggtggcaaagaatacaaacaacaagatcagccggctgctggactccctcccttctgacacaaggctggtcctcctgaatgccatctacctgtctgccaagtggaagaccacatttgatcccaaaaagacaagaatggaaccattccactttaagaactccgtgatcaaagtccccatgatgaacagcaagaagtaccctgtggcccacttcattgatcagaccctgaaagccaaagtgggccagctgcagctttcccacaacctgtccttggtcatcctggtgccccagaacctcaagcaccgcctggaagatatggaacaagcactcagcccatctgtcttcaaggccattatggagaaactggagatgtccaaattccagcccactctcctcaccctgcccagaatcaaggtgactaccagccaagacatgctgagcatcatggagaagctcgagttctttgacttctcctatgacctcaacttgtgtggcctgactgaggaccctgatctgcaagtcagtgccatgcagcaccagacagtgttggagctgacagaaactggtgtggaagctgctgcagcctctgctatctcagtggctcggaccttgcttgtgtttgaggtgcagcaacccttcctgtttgtcctgtgggaccagcagcataagtttccagtgttcatgggccgggtgtatgatccaagagcctga

SEQ ID NO: 18 - DNA, 218 bp, Artificial Sequence
  3X CRM8
gggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccac

SEQ ID NO: 19 - DNA, 77 bp, Artificial Sequence
  MVM Intron
ctaaggtaagttggcgccgtttaagggatggttggttggtggggtattaatgtttaattaccttttttacaggcctg

SEQ ID NO: 20 - DNA, 717 bp, Artificial Sequence
  SERPING1 exon1-intron1-partially exon2
ctgatttacaggaactcacaccagcgatcaatcttccttaatttgtaactgggcagtgtcccgggccagccaatagctaagactgccccccccgcaccccaccctccctgaccctgggggactctctactcagtctgcactggagctgcctggtgaccagaagtttggagtaggtttggtgctgggcaggggtggggagtagggtggaaagcatggagtgaagaggtctagggagggggtctcctcacccccgccttcctgcccgccttgatctcgggggtctctataggcttgcttccacctgggacttctgcctcctcctaccccagcccctcccgcctcaggcctgttgtgctcagccccccaggacctcccctcccccacgcctctggcctcattgtttggttaaagcaggaccccctccccctcccaccacctcccctccgactgaacagatggacagagacccgggcccacggggagaggaagggccagccggtgccgggaaagggaagcggtttggggaaaacaaaacagagggaggagccagggagaaggtggccccaggagggaggaggagggaattcgctaagagggactggggcctgagacggaatgggggcgggccccgggcggggtgggggcccctgggctcccagggtgggagctggctccgaggctggctggctccgcaggtccgctgacgtcgccgcccag

SEQ ID NO: 21 - DNA, 234 bp, Artificial Sequence
  BGHpA
cctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaa

SEQ ID NO: 22 - DNA, 592 bp, Artificial Sequence
  WPRE WT
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 23 - DNA, 248 bp, Artificial Sequence
  WPRE3
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtt

SEQ ID NO: 24 - DNA, 602 bp, Artificial Sequence
  WPREmut6delATG
gataatcaacctctggattacaaaatttgtgaaagattgactggtattcttaactttgttgctccttttacgctttgtggatacgctgctttattgcctttgtatcttgctattgcttcccgtttggctttcattttctcctccttgtataaatcctggttgctgtctctttttgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactag

SEQ ID NO: 25 - DNA, 426 bp, Artificial Sequence
  CRE4
gagttaatgtgtaatgagacggggagaaaacaggagagcccagaatgacctggatgctgatcactgaacataccctacccccagtaaaacaaattcagaaaacagcttccgcccgtcccctcccaatggagggctctggcaggaaaagaggtgaataagaggcttctaccagggtaaaggttgaaggcacctggtcatttgatcaccttatcagttctaggcagtgattagccaatattgagtcagcaggggcaatagccctggcccttgtctcactcctgttgggggtgggggagggggagaggtacattcccaggttcaaagcatttgggtgaaatcagttaaatagatatcagaagcttttgtatctttcacccttttgccccccaagcatactcgctgagtatgtggaacattcctgagggt

SEQ ID NO: 26 - DNA, 267 bp, Artificial Sequence
  CRE6
aggggggcccctggggctgacagggactggaagctctgagctggccagagggatgttgcaatcctgccagggtcttgtctatgctgtccttttcacaaccatccccctactgccaggctgacacgtggttgcgggggcacaaggccagccaacctagagtctgaggctaggcggaggacaccctccccaccagctgccagggtcactggcggtcaaaggcagctggtggggaaggcattggactccagccttgggggacggatgtag

SEQ ID NO: 27 - DNA, 233 bp, Artificial Sequence
  hTTR
aaatgacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttggcagg

SEQ ID NO: 28 - DNA, 222 bp, Artificial Sequence
  mod_mTTR
gtctgtctgcacatttcgtagagcgagtgttccgatactctaatctccctaggcaaggttcatattgacttaggttacttattctccttttgttgactaagtcaataatcagaatccagcaggtttggagtcagcttggcagggatcagcagccttgggttggaaggagggggtataaaagccccttcaccaggagaagccgtcacacagatccacaagctc

SEQ ID NO: 29 - DNA, 229 bp, Artificial Sequence
  WT mTTR
ggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacaga

(19) United States
(12) Patent Application Publication
(10) Pub. No.: US 2023/0043051 A1
(43) Pub. Date: Feb. 9, 2023

(54) ADENO-ASSOCIATED VIRUS VECTORS BASED GENE THERAPY FOR HEREDITARY ANGIOEDEMA

(71) Applicant: Shire Human Genetic Therapies, Inc., Lexington, MA (US)

(72) Inventors: Matthias Klugmann, Lexington, MA (US); Franziska Horling, Lexington, MA (US); Johannes Lengler, Lexington, MA (US); Patrice Douillard, Lexington, MA (US); Friedrich Scheiflinger, Lexington, MA (US); Hanspeter Rottensteiner, Lexington, MA (US); Bagirath Gangadharan, Lexington, MA (US)

(21) Appl. No.: 17/771,275

(22) PCT Filed: Oct. 23, 2020
(86) PCT No.: PCT/US2020/056984

§ 371 Date: Apr. 22, 2022

Related U.S. Application Data
(60) Provisional application No. 62/924,877, filed on Oct. 23, 2019.

(51) Int. Cl. C12N 15/861; C07K 14/81; A61P 7/10

(52) U.S. Cl. CPC C12N 15/861; C07K 14/8121; A61P 7/10; C12N 2750/14143; C12N 2830/008; C12N 2830/42; C12N 2830/48

(57) ABSTRACT

The present disclosure provides, among other things, a recombinant adeno-associated virus (rAAV) vector comprising an AAV8 capsid and a codon-optimized SERPING1 sequence encoding a human C1-esterase inhibitor. The disclosure also provides a method of treating a subject having Hereditary angioedema (HAE), comprising administering to the subject in need thereof a recombinant adeno-associated virus (rAAV) vector comprising an AAV8 capsid, and codon-optimized SERPING1 sequences encoding a human C1-esterase inhibitor.

### CROSS-REFERENCE TO RELATED APPLICATIONS

The present application claims priority to U.S. Provisional Application Ser. No. 62/924,877, filed Oct. 23, 2019, the disclosure of which is hereby incorporated by reference in its entirety.

### BACKGROUND

Hereditary angioedema (HAE) is a rare condition characterized by recurrent episodes of swelling in face, throat, and in most extremities. HAE is a potentially life-threatening disorder characterized by unpredictable and recurrent attacks of vasodilation manifesting as subcutaneous and submucosal angioedema. In some cases, HAE is associated with low plasma levels of C1-inhibitor (type I), while in other cases the protein circulates in normal or elevated amounts but it is dysfunctional (type II). C1 inhibitor is the main regulator of plasma kallikrein activity. Symptoms of HAE attacks include swelling of the face, mouth and/or airway that occur spontaneously or are triggered by mild trauma. Edematous attacks affecting the airways can be fatal. In addition to acute inflammatory flares, excess plasma kallikrein activity has also been associated with chronic conditions, such as autoimmune diseases, including lupus erythematosus.

Various strategies for the treatment of C1-INH deficiencies or dysfunctions have been contemplated and developed, including for example inhibiting members of the contact system. For example, lanadelumab is a fully human monoclonal antibody inhibitor of plasma kallikrein that has been approved for the treatment of HAE.

Use of vectors that produce proteins in vivo is desirable for the treatment of disease, but is limited by various factors including poor protein production following delivery to a subject.

To date, available therapies have not addressed challenges including disease recurrence and the need for long-term continued administration. Thus, there is a need for a novel and lasting therapeutic approach to treat HAE.

### SUMMARY

The present invention provides recombinant adeno-associated virus (rAAV) vectors that allow for efficient and robust human C1 esterase inhibitor (C1-INH or C1EI) expression in vivo.

In one aspect, the present invention provides, among other things, a recombinant adeno-associated virus (rAAV) vector including an AAV8 capsid and a codon-optimized SERPING1 sequence encoding a C1 inhibitor (C1-INH).

In some embodiments, the codon-optimized SERPING1 sequence encoding C1-INH includes a sequence having at least about 70%, 75%, 80%, 85%, 90%, 95% or 99% identity to SEQ ID NO: 2.

In some embodiments, the codon-optimized SERPING1 sequence encoding C1-INH includes a sequence identical to SEQ ID NO: 2.

In some embodiments, the vector further includes a liver-specific promoter.

In some embodiments, the liver-specific promoter is transthyretin promoter (TTR).

In some embodiments, the vector further includes a ubiquitous promoter.

In some embodiments, the vector further includes one or more of the following: a 5′ and a 3′ inverted terminal repeat, an intron upstream of the sequence, and a cis-acting regulatory module (CRM).

In some embodiments, the vector further comprises a WPRE sequence.

In some embodiments, the WPRE sequence is modified.

In some embodiments, the WPRE contains a mut6delATG modification.

In some embodiments, the intron is a minute virus of mice (MVM) or SV40 intron.

In some embodiments, the CRM is liver-specific CRM.

In some embodiments, the CRM is CRM8.

In some embodiments, the vector comprises at least three CRMs.

In another aspect, the present invention provides, among other things, a recombinant adeno-associated virus (rAAV) including an AAV8 capsid and an rAAV vector, and vector including:

a. a 5′ inverted terminal repeat (ITR);

b. a cis-acting regulatory module (CRM);

c. a liver specific promoter;

d. a minute virus of mice (MVM);

e. a SERPING1 sequence encoding C1 inhibitor (C1-INH);

f. a woodchuck hepatitis virus posttranscriptional regulatory element (WPRE); and

g. a 3′ ITR.

In some embodiments, the SERPING1 sequence is a wild type sequence or a codon-optimized sequence.

In some embodiments, the codon-optimized SERPING1 sequence has at least about 70%, 75%, 80%, 85%, 90%, 95%, or 99% identity with SEQ ID NO: 2.

In another aspect, the present invention provides, among other things, a method of treating a subject having hereditary angioedema (HAE), comprising administering to the subject in need thereof a rAAV of any one of the preceding embodiments.

In another aspect, the present invention provides, among other things, a method of treating a subject having hereditary angioedema (HAE), comprising administering to the subject in need thereof a recombinant adeno-associated virus (rAAV) vector comprising an AAV8 capsid, and a promoter operably linked to a nucleic acid sequence that encodes C1 inhibitor (C1-INH), and wherein administering results in an increase in C1-INH enzymatic activity in the subject.

In some embodiments, C1-INH is detected in the plasma of the subject.

In some embodiments, C1-INH is detected in the liver of the subject.

In some embodiments, C1-INH is maintained for at least 30, 60, 90, 120, 150, 180 days or more after a single administration.

In some embodiments, C1-INH activity is present in the subject following administration of the rAAV vector.

In some embodiments, the subject has C4 level restored to a pre-attack level.

In some embodiments, the AAV is administered intravenously.

In some embodiments, the AAV is administered intrathecally.

In some embodiments, the AAV is administered at dose of at least about 5×10⁹ vg.

In some embodiments, the administering of the rAAV does not elicit immune response.

Various aspects of the invention are described in detail in the following sections. The use of sections is not meant to limit the invention. Each section can apply to any aspect of the invention. In this application, the use of “or” means “and/or” unless stated otherwise. As used herein, the singular forms “a”, “an”, and “the” include both singular and plural referents unless the context clearly dictates otherwise.

### BRIEF DESCRIPTION OF THE DRAWINGS

FIG. 1A is a schematic representation of the expression cassette comprising wild-type, human C1-INH (hC1-INH) expression vector. FIG. 1B is a schematic representation of the expression cassette designed for hC1-INH expression in liver (hepatocytes). ITR inverted terminal repeat; hTTR: human transthyretin promoter; CRM: cis-acting regulatory module; intron can be a MVM intron (minute virus of mice intron); and polyA is an upstream enhancer.

FIG. 2A is a general schematic representation of a M construct. M construct is a control construct having HA01 (SERPING1) sequence. HA01 is a human SERPING1 wild-type sequence having 1503 bp, 24 CpG's, and 53.4% GC content.

FIG. 2B shows two M constructs: M01 and M01A. M01 includes HA01 (SERPING1) sequence and Exon 1-Intron 1-partially Exon 2 (717 bp) intron, whereas Mar01A includes HA01 (SERPING1) sequence and MVM (77 bp) intron.

FIG. 3A is a general schematic representation of a J construct. J construct includes four codon-optimized SERPING1 sequences. FIG. 3B shows schematic representations of four J constructs—J01, J02, J03, and J04. J01 includes codon-optimized SERPING1 sequence, HA03. J02 includes codon-optimized SERPING1 sequence, HA06. J03 includes codon-optimized SERPING1 sequence, HA05. J04 includes codon-optimized SERPING1 sequence, HA04.

FIG. 4A is a general schematic representation of a S construct. All S constructs includes a codon-optimized HA06 (SERPING1) sequence different combinations of introns and WPREs. Introns includes either a MVM intron or a Exon 1-Intron 1-partially Exon 2. FIG. 4B shows schematic representation of nine S constructs—S01, S02, S03, S04, S05, S06, S07, S08, and S09.

FIG. 5A is a general schematic representation of an N construct. All N constructs includes codon-optimized HA06 (SERPING1) sequence and novel promoter elements. FIG. 5B shows schematic representation of nine N constructs—N01, N02, N03, N04, N05, N06, N07, N08, and N09.

FIG. 6A is a general schematic representation of a U construct. U construct includes different codon-optimized SERPING1 sequences in combination with WPREmut6delATG. FIG. 6B shows schematic representation of ten U constructs—U01, U02, U03, U04, U05, U06, U07, U08, U09, and U10. U01 includes codon-optimized SERPING1 sequence, HA11. U02 includes codon-optimized SERPING1 sequence, HA12. U03 includes codon-optimized SERPING1 sequence, HA13. U04 includes codon-optimized SERPING1 sequence, HA14. U05 includes codon-optimized SERPING1 sequence, HA15. U06 includes codon-optimized SERPING1 sequence, HA16. U07 includes codon-optimized SERPING1 sequence, HA17. U08 includes codon-optimized SERPING1 sequence, HA18. U09 includes codon-optimized SERPING1 sequence, HA19. U10 includes codon-optimized SERPING1 sequence, HA20.

FIG. 7A is a general schematic representation of a P construct. All P constructs includes a codon-optimized HA06 (SERPING1) sequences and WPREmut6delATG, in combination with different novel promoter elements. The novel promotor elements include a CRE4, CRE6, or CRE4 and CRE6. FIG. 7B shows schematic representation of six P constructs—P01, P02, P03, P04, P05, and P06.

FIG. 8A is an image of a Western blot which depicts expression of hC1-INH by AAV8.SERPING1 vector in HepG2 cells. FIG. 8B is a graph of an experimental data illustrating the expression of functional C1-INH by AAV8.SERPING1 in HepG2 cells, as measured by an ELISA assay.

FIG. 9A is a graph of experimental data illustrating a dose dependent expression of hC1-INH following a single intravenous administration of the AAV8.SERPING1 vector to wild-C57B1/6Albino mice. The level of hC1-INH was measured in plasma at day 14 post injection. FIG. 9B is a graph of experimental data illustrating a dose dependent expression of hSERPING1 DNA or RNA following a single intravenous administration of the AAV8.SERPING1 vector to wild-C57Bl/6Albino mice. The level of hC1-INH was measured in liver at day 28 post injection. Level of expression of hSERPING1 DNA is presented in terms of copies/μg as well as copies/cell.

FIG. 10A is a graph of experimental data illustrating in vivo efficacies of rAAV8 vectors comprising four codon-optimized SERPING1 constructs (J01, J02, J03, and J04) in expressing C1-INH in plasma following a single intravenous administration of different vectors to wild-C57Bl/6Albino mice. The level of expression of hC1-INH was measured at day 7, 14 and 28 post injection. M01A construct was used as a control. Schematic representations of different constructs used in this study are shown in FIG. 10B.

FIG. 11 is a graph of experimental data illustrating in vivo efficacies of rAAV8 vectors comprising codon-optimized SERPING1 sequences and WPREs in expressing C1-INH in plasma following a single intravenous administration of vectors to wild-C57Bl/6Albino mice. The level of expression of hC1-INH was measured at day 7, 14 and 28 post injection. A single vector dose 4×10¹¹ vg/kg was administered.

FIG. 12A is a graph of experimental data illustrating in vivo efficacies of rAAV8 vectors comprising HA06 (SERPING1) sequence, different WPREs and introns, in expressing C1-INH in plasma following a single intravenous administration of vectors to C57Bl/6Albino mice. The level of expression of hC1-INH was measured at day 7, 14 and 28 post injection. Schematic representations of different constructs used in this study are shown in FIG. 12B.

FIG. 13A is a graph of experimental data illustrating in vivo efficacies of rAAV8 vectors comprising HA06 (SERPING1) sequence, WPRE and different promoter elements, in expressing C1-INH in plasma following a single intravenous administration of vectors at a dose of 4×10¹¹ vg/kg to C57Bl/6Albino mice. The level of expression of hC1-INH was measured at day 7, 14 and 28 post injection. Schematic representations of different constructs used in this study are shown in FIG. 13B. FIG. 13C is a graph of experimental data illustrating dose dependent efficacy of S04, a construct comprising HA06 (SERPING1) sequence, WPRE mut6delATG and MVM intron, in expressing C1-INH in plasma following intravenous administration of the construct at low (2×10¹² vg/kg) and high (2×10¹³ vg/kg) doses to C57Bl/6Albino mice. The level of expression of hC1-INH was measured at day 14 and day 28 post injection. Schematic representation of S04 is shown in FIG. 13C.

FIG. 14A is a graph of experimental data illustrating in vivo efficacies of rAAV8 vectors comprising codon-optimized SERPING1 sequences in expressing C1-INH in plasma following a single intravenous administration of vectors at a dose of 2×10¹² vg/kg to C57Bl/6Albino mice. The level of expression of hC1-INH was measured at day 28 post injection. The level of expression of the vectors comprising codon-optimized SERPING1 sequences were compared with that of a S04 construct at the same dose. Schematic representations of different constructs used in this study are shown in FIG. 14B.

FIG. 15A is a graph of experimental data illustrating in vivo efficacies of rAAV8 vectors comprising HA06 (SERPING1) sequence in combination with different WPREs and novel promoter elements, in expressing C1-INH in plasma following a single intravenous administration of a medium dose of vectors to C57Bl/6Albino mice. The level of expression of hC1-INH was measured at day 7, 14, 28, 49, 70, 91, 112, 133, 152, and 175 post injection. Schematic representations of different constructs used in this study are shown in FIG. 15B.

### DEFINITIONS

Adeno-associated virus (AAV): As used herein, the terms “adeno-associated virus” or “AAV” or recombinant AAV (“rAAV”) includes, but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV (see, e.g., Fields et al., Virology, volume 2, chapter 69 (4th ed., Lippincott-Raven Publishers); Gao et al., J. Virology 78:6381-6388 (2004); Mori et al., Virology 330:375-383 (2004)). Typically, AAV can infect both dividing and non-dividing cells and can be present in an extrachromosomal state without integrating into the genome of a host cell. AAV vectors are commonly used in gene therapy.

Administering: As used herein, the terms “administering,” or “introducing” are used interchangeably in the context of delivering rAAV vectors encoding a therapeutic into a subject, by a method or route which results in delivery of the rAAV vector. Various methods are known in the art for administering rAAV vectors, including for example intravenously, subcutaneously or transdermally. Transdermal administration of rAAV vector can be performed by use of a “gene gun” or biolistic particle delivery system. In some embodiments, the rAAV vectors are administered via non-viral lipid nanoparticles.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Functional equivalent or derivative: As used herein, the term “functional equivalent” or “functional derivative” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

IRES: As used herein, the term “IRES” refers to any suitable internal ribosome entry site sequence.

Polypeptide: The term, “polypeptide,” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Regulatory element: As used herein, the term “regulatory element” refers to transcriptional control elements, in particular non-coding cis-acting transcription control elements, capable of regulating and/or controlling transcription of a gene. Regulatory elements comprise at least one transcription factor binding site, for example at least one binding site for a tissue specific transcription factor. In embodiments described herein, regulatory elements have at least one binding site for a liver-specific transcription factor. Typically, regulatory elements increase or enhance promoter-driven gene expression when compared to the transcription of the gene from the promoter alone, without the regulatory elements. Thus, regulatory elements particularly comprise enhancer sequences, although it is to be understood that the regulatory elements enhancing transcription are not limited to typical far upstream enhancer sequences, but may occur at any distance of the gene they regulate. As is understood in the art, sequences regulating transcription may be situtated either upstream (e.g., in the promoter region) or downstream (e.g., in the 3′UTR) of the gene that is regulated in vivo, and may be located in the immediate vicinity of the gene or further away. Regulatory elements can comprise either naturally occurring sequences, combinations of (parts of) such regulatory elements or several copies of a regulatory element, e.g., non-naturally occurring sequences. Accordingly, regulatory elements include naturally occurring and optimized or engineered regulatory elements to achieve a desired expression level.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a “homologous” substitution.

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., basic local alignment search tool, J. Mol. Biol., 215(3): 403-410, 1990; Altschul, et al., Methods in Enzymology, Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, 55%, 60%, 65%, 70%, 75%, 80%, 85%, 90%, 91%, 92%, 93%, 94%, 95%, 96%, 97%, 98%, 99% or more of their corresponding residues are homologous over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990: Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Suffering from: An individual who is “suffering from” a disease, disorder, and/or condition has been diagnosed with or displays one or more symptoms of the disease, disorder, and/or condition.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

The recitation of numerical ranges by endpoints herein includes all numbers and fractions subsumed within that range (e.g. 1 to 5 includes 1, 1.5, 2, 2.75, 3, 3.9, 4 and 5). It is also to be understood that all numbers and fractions thereof are presumed to be modified by the term “about.”

Various aspects of the invention are described in detail in the following sections. The use of sections is not meant to limit the invention. Each section can apply to any aspect of the invention. In this application, the use of “or” means “and/or” unless stated otherwise. As used herein, the singular forms “a”, “an”, and “the” include both singular and plural referents unless the context clearly dictates otherwise.

Various aspects of the invention are described in detail in the following sections. The use of sections is not meant to limit the invention. Each section can apply to any aspect of the invention. In this application, the use of “or” means “and/or” unless stated otherwise.

### DETAILED DESCRIPTION

The present disclosure describes efficient and robust recombinant adeno-associated virus (rAAV) vectors for in vivo production of C1-INH for the treatment of diseases associated with a C1-INH deficiency, such as HAE.

### Heredetary Angioedema (HAE)

HAE is characterized by decreased levels of C1-INH with concomitant upregulation of bradykinin. It is inherited in an autosomal dominant pattern and affects 1:10,000 to 1:50,000 people. The underlying cause of HAE (type I and II) is attributed to autosomal dominant inheritance of mutations in the C1 esterase inhibitor gene (C1EI gene or SERPING1 gene), mapped to chromosome 11. Autosomal dominant mutations in the SERPING1 gene can be more than 300, yet it is amenable to gene therapy. Eighty-five percent of HAE cases are type I in which there is a deficiency in the amount of C1 esterase inhibitor produced (see e.g., Gower et al., World Allergy Organ J., 4: S9-S21 (2011); Cungo et al., Trends Mol Med, 15: 69-78 (2009); Gooptu et al., Annu Rev Biochem, 78: 147-176 (2009); and Zuraw et al., J Allergy Clin Immunol Pract, 1: 458-467 (2013)). The remainder of cases are characterized by the expression of a dysfunctional C1 esterase inhibitor.

The frequency, duration and severity of attacks associated with HAE vary, with 30% of patients reporting a frequency of greater than one attack/month, 40% report 6 to 11 attacks/year and the remaining 30% are infrequently symptomatic. Usually, symptoms are transient progressing over 12 to 36 hours and subsiding within 2 to 5 days; however, some attacks may last up to one week. Although HAE episodes are self-limiting, the unpredictable occurrence of attacks places considerable strain on patients, often heavily impacting quality of life, and can be fatal.

To date, therapeutic agents are indicated for long-term prophylaxis, therapy for acute attacks and short-term prophylaxis (i.e., prior to dental surgery), and include agents such as Danazol, which has a high adverse effect profile, C1 inhibitor replacement protein, bradykinin receptor antagonists, kallikrein inhibitors, fresh frozen plasma and purified C1 inhibitor. These therapies can alleviate symptoms and maximize quality of life; however, disease recurrence and the need for long-term continued administration remains a major obstacle to therapy (see e.g., Aberer, Ann Med, 44: 523-529 (2012); Charignon et al., Expert Opin Pharmacother, 13: 2233-2247 (2012); Papadopoulou-Alataki, Curr Opin Allergy Clin Immunol, 10: 20-25 (2010); Parikh et al., Curr Allergy Asthma Rep, 11: 300-308 (2011); Tourangeau et al., Curr Allergy Asthma Rep, 11: 345-351 (2011); Bowen et al., Ann Allergy Asthma Immunol, 100: S30-S40 (2008); Frank, Immunol Allergy Clin North Am, 26: 653-668 (2006); Cicardi et al., J Allergy Clin Immunol, 99: 194-196 (1997); Kreuz et al., Transfusion 49: 1987-1995 (2009); Bork et al., Ann Allergy Asthma Immunol, 100: 153-161 (2008); and Cicardi et al., J Allergy Clin Immunol, 87: 768-773 (1991)).

The present invention provides, among other things, methods and compositions for treating HAE using recombinant adeno-associated virus (rAAV) vectors including codon-optimized SERPING1 sequence that encode hC1-INH. In particular, the present invention provides a method of treating HAE by administering a rAAV comprising a codon-optimized sequence SERPING1 encoding a human C1-INH at an effective dose such that at least one symptom or feature of HAE is reduced in intensity, severity, or frequency. The gene therapy method described herein was particularly effective in expressing therapeutic level of hC1-INH.

rAAV SERPING1 Vector Design

In some aspects, provided herewith is a recombinant adeno-associated virus (rAAV) vector encoding C1-INH. The rAAV vector comprises a capsid and a SERPING1 sequence.

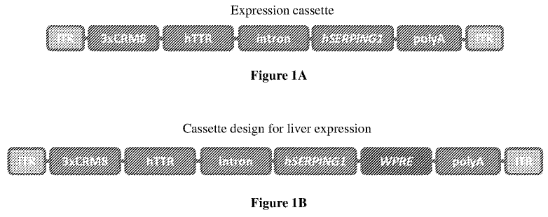

Schematic that illustrate exemplary rAAV vectors of the present disclosure are illustrated in FIG. 1A and FIG. 1B. As shown in FIG. 1A, in some embodiments, a rAAV vector of the present disclosure comprises a liver specific promoter, a 5′ and a 3′ inverted terminal repeat (ITR), a cis-acting regulatory module (CRM), and an intron.

The SERPING1 sequence of the vector can be a wild-type or a codon-optimized variant. Accordingly, in some embodiments, the rAAV vector comprises a wild-type SERPING1 nucleotide sequence. In some embodiments, the rAAV vector comprises a codon-optimized SERPING1 sequence.

A suitable C1-INH of the present invention is any protein or a portion of a protein that can substitute for at least partial activity of naturally-occurring C1-INH or rescue one or more phenotypes or symptoms associated with C1-INH-deficiency.

In some embodiments, a suitable C1-INH nucleotide sequence for the present invention comprises a SERPING1 sequence encoding human C1-INH protein. The naturally-occurring human C1-INH nucleotide sequence is shown in GenBank: AF435921.1. The corresponding human C1-INH amino acid sequence is shown in Table 1:

TABLE 1
Human C1-INH Sequence
HUMAN C1-INH (AMINO ACID SEQUENCE)
(GenBank: AAM21515.1)

MASRLTLLTLLLLLLAGDRASSNPNATSSSSQDPESLQDRGEGKVATT
VISKMLFVEPILEVSSLPTTNSTTNSATKITANTTDEPTTQPTTEPTT
QLPTDSPTQPTTGSFCPGPVTLCSDLESHSTEAVLGDALVDFSLKLYH
AFSAMKKVETNMAFSPFSIASLLTQVLLGAGENTKTNLESILSYPKDF
TCVHQALKGFTTKGVTSVSQIFHSPDLAIRDTFVNASRTLYSSSPRVL
SNNSDANLELINTWVAKNTNNKISRLLDSLPSDTRLVLLNAIYLSAKW
KTTFDPKKTRMEPFHFKNSVIKVPMMNSKKYPVAHFIDQTLKAKVGQL
QLSHNLSLVILVPQNLKHRLEDMEQAPSPSVFKAIMEKLEMSKFQPTL
LTLPRIKVTTSQDMLSIMEKLEFFDFSYDLNLCGLTEDPDLQVSAMQH
QTVLELTETGVEAAAASAISVARTLLVFEVQQPFLFVLWDQQHKFPVF
MGRVYDPRA
(SEQ ID NO: 1)

Various kinds of promoters can be used in the rAAV vector described herein. These include, for example, ubiquitous, tissue-specific, and regulatable (e.g. inducible or repressible) promoters. In some embodiments, the promoter is a liver-specific promoter. Examples of liver-specific promoters are known in the art and include, for example, human transthyrethin promoter (TTR), modified hTTR, (hTTR mod.), α-Antitrypsin promoter, human factor IX pro/liver transcription factor-responsive oligomers, LSP, CMV/CBA promoter (1.1 kb), CAG promoter (1.7 kb), mTTR, modified mTTR, mTTR pro, mTTR enhancer, and the basic albumin promoter. Liver specific promoters are described, for example, in Zhijian Wu et al., Molecular Therapy vol. 16, no 2, February 2008, the contents of which are incorporated herein by reference.

In some embodiments, the promotor is a ubiquitous promoter. In some embodiments, the promoter is a chicken beta actin promoter.

In some embodiments, the rAAV vector contains additional enhancer or regulatory elements to promote transcription and/or translation of the mRNA (e.g., enhancer sequences, Kozak sequences, polyadenylation sequences, transcriptional termination sequences, IRES and the like). In some embodiments, the vector comprises a 5′ and a 3′ inverted terminal repeat (ITR). In some embodiments, the vector comprises one or more enhancer elements. In some embodiments, the vector comprises a polyA tail.

In some embodiments, the rAAV vector comprises one or more small elements, such as an intron. Various introns are known in the art. Suitable introns for the rAAV vector described herein include for example an MVM intron, a truncated F.IX intron, a chimeric β globin SD/immunoglobulin heavy chain SA intron, SV40 and/or an alpha globin 1^{st} intron. In some embodiments, the rAAV vector comprises an MVM intron. In some embodiments, the rAAV vector comprises an SV40 intron. In some embodiments, the intron can be an Exon1-Intron1-partially Exon2 from the SERPING1 gene.

In some embodiments, the rAAV vector comprises woodchuck hepatitis virus post-transcriptional control element (WPRE) as WPRE increases transgene expression of viral vectors in a host of tissues. Various optimized or variant forms of WPRE are known in the art, and include for example WPRE wild-type, WPRE3, and WPREmut6delATG among others. WPRE and associated WPRE variants are described in U.S. Pat. Nos. 10,179,918; 7,419,829; 9,731,033; 8,748,169; 7,816,131; 8,865,881; 6,287,814; U.S. Patent Publication No. 2016/0199412; U.S. Patent Publication No. 2017/0114363; U.S. Patent Publication No. 2017/0360961; U.S. Patent Publication No. 2019/0032078; U.S. Patent Publication No. 2018/0353621; International Publication No. WO2017201527; International Publication No. WO2018152451; International Publication No. WO2013153361; International Publication No. WO2014144756; European Patent No. EP1017785; and European Patent Publication No. 3440191. Each of the foregoing publications are incorporated herein by reference in its entirety.

In some embodiments, the rAAV vector comprises one or more cis regulatory elements (CREs). CREs are modified backbone elements of a construct. Various optimized or variant forms of CRE are known in the art, and include for example CRE4 and CRE6 among others. The following publications describe different variants of CRE, and each of which is incorporated herein by reference: International Publication Nos. WO2016146757, WO2014064277, WO2014063753, and WO2009130208.

In some embodiments, the rAAV vector comprises a cis-actin regulatory module (CRM). Various kinds of CRM are suitable for use in the vectors described herein and include for example liver-specific CRM, neuronal-specific CRM and/or CRM8. In some embodiments, the vector includes more than one CRM. For example, in some embodiments, the vector comprises two, three, four, five or six CRM. In some embodiments, the vector comprises three CRM, for example three CRM8.

In some embodiments, the rAAV vector is sequence optimized to increase transcript stability, for more efficient translation, and to reduce immunogenicity. In some embodiments, the rAAV vector is sequence optimized to increase transcript stability, for more efficient translation, and/or to reduce immunogenicity. In some embodiments, the SERPING1 is sequence optimized.

In some embodiments, the rAAV vector is an AAV1, AAV2, AAV3, AAV4, AAV5, AAV6, AAV7, AAV8, AAV9, AAV10, or AAV11 vector. In some embodiments, the rAAV vector is AAV1. In some embodiments, the rAAV vector is AAV 2. In some embodiments, the rAAV vector is AAV 3. In some embodiments, the rAAV vector is AAV 4. In some embodiments, the rAAV vector is AAV 5. In some embodiments, the rAAV vector is AAV 6. In some embodiments, the rAAV vector is AAV 7. In some embodiments, the rAAV vector is AAV 8. In some embodiments, the rAAV vector is AAV 9. In some embodiments, the rAAV vector is AAV 10. In some embodiments, the rAAV vector is AAV 11. In some embodiments, the rAAV vector is sequence optimized. In some embodiments, the rAAV capsid is modified

Exemplary element sequences are shown in Table 2 below. In some embodiments, the rAAV vector comprises a rAAV vector element comprising a nucleotide sequence having at least 60%, 65%, 70%, 75%, 80%, 85%, 90%, 95% or 99% identity with a vector element sequence shown in Table 2. In some embodiments, the rAAV vector comprises a vector element nucleotide sequence identical to a vector element nucleotide sequence shown in Table 2.

TABLE 2
Sequences that are contained in the HAE constructs (expression cassette)
of the vector
SERPING1 wild type sequence (53% GC content, 24 CPG’s)
(NCBI Reference Sequence: NM_000062.2)
ATGGCCTCCAGGCTGACCCTGCTGACCCTCCTGCTGCTGCTGCTGGCTGGGGATAGAGCCTCC
TCAAATCCAAATGCTACCAGCTCCAGCTCCCAGGATCCAGAGAGTTTGCAAGACAGAGGCGAA
GGGAAGGTCGCAACAACAGTTATCTCCAAGATGCTATTCGTTGAACCCATCCTGGAGGTTTCC
AGCTTGCCGACAACCAACTCAACAACCAATTCAGCCACCAAAATAACAGCTAATACCACTGAT
GAACCCACCACACAACCCACCACAGAGCCCACCACCCAACCCACCATCCAACCCACCCAACCA
ACTACCCAGCTCCCAACAGATTCTCCTACCCAGCCCACTACTGGGTCCTTCTGCCCAGGACCT
GTTACTCTCTGCTCTGACTTGGAGAGTCATTCAACAGAGGCCGTGTTGGGGGATGCTTTGGTA
GATTTCTCCCTGAAGCTCTACCACGCCTTCTCAGCAATGAAGAAGGTGGAGACCAACATGGCC
TTTTCCCCATTCAGCATCGCCAGCCTCCTTACCCAGGTCCTGCTCGGGGCTGGGGAGAACACC
AAAACAAACCTGGAGAGCATCCTCTCTTACCCCAAGGACTTCACCTGTGTCCACCAGGCCCTG
AAGGGCTTCACGACCAAAGGTGTCACCTCAGTCTCTCAGATCTTCCACAGCCCAGACCTGGCC
ATAAGGGACACCTTTGTGAATGCCTCTCGGACCCTGTACAGCAGCAGCCCCAGAGTCCTAAGC
AACAACAGTGACGCCAACTTGGAGCTCATCAACACCTGGGTGGCCAAGAACACCAACAACAAG
ATCAGCCGGCTGCTAGACAGTCTGCCCTCCGATACCCGCCTTGTCCTCCTCAATGCTATCTAC
CTGAGTGCCAAGTGGAAGACAACATTTGATCCCAAGAAAACCAGAATGGAACCCTTTCACTTC
AAAAACTCAGTTATAAAAGTGCCCATGATGAATAGCAAGAAGTACCCTGTGGCCCATTTCATT
GACCAAACTTTGAAAGCCAAGGTGGGGCAGCTGCAGCTCTCCCACAATCTGAGTTTGGTGATC
CTGGTACCCCAGAACCTGAAACATCGTCTTGAAGACATGGAACAGGCTCTCAGCCCTTCTGTT
TTCAAGGCCATCATGGAGAAACTGGAGATGTCCAAGTTCCAGCCCACTCTCCTAACACTACCC
CGCATCAAAGTGACGACCAGCCAGGATATGCTCTCAATCATGGAGAAATTGGAATTCTTCGAT
TTTTCTTATGACCTTAACCTGTGTGGGCTGACAGAGGACCCAGATCTTCAGGTTTCTGCGATG
CAGCACCAGACAGTGCTGGAACTGACAGAGACTGGGGTGGAGGCGGCTGCAGCCTCCGCCATC
TCTGTGGCCCGCACCCTGCTGGTCTTTGAAGTGCAGCAGCCCTTCCTCTTCGTGCTCTGGGAC
CAGCAGCACAAGTTCCCTGTCTTCATGGGGCGAGTATATGACCCCAGGGCCTGA
(SEQ ID NO: 2)
human SERPING1 wild type sequence (different stop codon than SEQ ID NO: 2)
HA01
(NCBI Reference Sequence: NM_000062.2)
ATGGCCTCCAGGCTGACCCTGCTGACCCTCCTGCTGCTGCTGCTGGCTGGGGATAGAGCCTCC
TCAAATCCAAATGCTACCAGCTCCAGCTCCCAGGATCCAGAGAGTTTGCAAGACAGAGGCGAA
GGGAAGGTCGCAACAACAGTTATCTCCAAGATGCTATTCGTTGAACCCATCCTGGAGGTTTCC
AGCTTGCCGACAACCAACTCAACAACCAATTCAGCCACCAAAATAACAGCTAATACCACTGAT
GAACCCACCACACAACCCACCACAGAGCCCACCACCCAACCCACCATCCAACCCACCCAACCA
ACTACCCAGCTCCCAACAGATTCTCCTACCCAGCCCACTACTGGGTCCTTCTGCCCAGGACCT
GTTACTCTCTGCTCTGACTTGGAGAGTCATTCAACAGAGGCCGTGTTGGGGGATGCTTTGGTA
GATTTCTCCCTGAAGCTCTACCACGCCTTCTCAGCAATGAAGAAGGTGGAGACCAACATGGCC
TTTTCCCCATTCAGCATCGCCAGCCTCCTTACCCAGGTCCTGCTCGGGGCTGGGGAGAACACC
AAAACAAACCTGGAGAGCATCCTCTCTTACCCCAAGGACTTCACCTGTGTCCACCAGGCCCTG
AAGGGCTTCACGACCAAAGGTGTCACCTCAGTCTCTCAGATCTTCCACAGCCCAGACCTGGCC
ATAAGGGACACCTTTGTGAATGCCTCTCGGACCCTGTACAGCAGCAGCCCCAGAGTCCTAAGC
AACAACAGTGACGCCAACTTGGAGCTCATCAACACCTGGGTGGCCAAGAACACCAACAACAAG
ATCAGCCGGCTGCTAGACAGTCTGCCCTCCGATACCCGCCTTGTCCTCCTCAATGCTATCTAC
CTGAGTGCCAAGTGGAAGACAACATTTGATCCCAAGAAAACCAGAATGGAACCCTTTCACTTC
AAAAACTCAGTTATAAAAGTGCCCATGATGAATAGCAAGAAGTACCCTGTGGCCCATTTCATT
GACCAAACTTTGAAAGCCAAGGTGGGGCAGCTGCAGCTCTCCCACAATCTGAGTTTGGTGATC
CTGGTACCCCAGAACCTGAAACATCGTCTTGAAGACATGGAACAGGCTCTCAGCCCTTCTGTT
TTCAAGGCCATCATGGAGAAACTGGAGATGTCCAAGTTCCAGCCCACTCTCCTAACACTACCC
CGCATCAAAGTGACGACCAGCCAGGATATGCTCTCAATCATGGAGAAATTGGAATTCTTCGAT
TTTTCTTATGACCTTAACCTGTGTGGGCTGACAGAGGACCCAGATCTTCAGGTTTCTGCGATG
CAGCACCAGACAGTGCTGGAACTGACAGAGACTGGGGTGGAGGCGGCTGCAGCCTCCGCCATC
TCTGTGGCCCGCACCCTGCTGGTCTTTGAAGTGCAGCAGCCCTTCCTCTTCGTGCTCTGGGAC
CAGCAGCACAAGTTCCCTGTCTTCATGGGGCGAGTATATGACCCCAGGGCCTAG
(SEQ ID NO: 3)
Codon-optimized SERPING1 sequences
HA03
ATGGCCAGCAGGCTGACCCTGCTGACCCTGCTGCTGCTGCTGCTGGCTGGAGACAGGGCCAGC
AGCAACCCCAATGCCACCAGCAGCAGCAGCCAGGACCCTGAGAGCCTGCAGGACAGGGGAGAG
GGCAAGGTGGCTACCACAGTGATCAGCAAGATGCTGTTTGTGGAGCCCATCCTGGAGGTGAGC
AGCCTGCCCACCACCAACAGCACCACCAACAGTGCCACCAAGATCACAGCCAACACCACAGAT
GAGCCCACCACCCAGCCCACCACAGAGCCCACCACCCAGCCCACCATCCAGCCCACCCAGCCC
ACCACCCAGCTGCCCACAGACAGCCCCACCCAGCCCACCACAGGCAGCTTCTGCCCTGGCCCT
GTGACCCTGTGCAGTGACCTGGAGAGCCACAGCACAGAGGCTGTGCTGGGAGATGCCCTGGTG
GACTTCAGCCTGAAGCTGTACCATGCCTTCAGTGCCATGAAGAAGGTGGAGACCAACATGGCC
TTCAGCCCCTTCAGCATTGCCAGCCTGCTGACCCAGGTGCTGCTGGGAGCTGGAGAGAACACC
AAGACCAACCTGGAGAGCATCCTGAGCTACCCCAAGGACTTCACCTGTGTGCACCAGGCCCTG
AAGGGCTTCACCACCAAGGGAGTGACCAGTGTGAGCCAGATCTTCCACAGCCCTGACCTGGCC
ATCAGGGACACCTTTGTGAATGCCAGCAGGACCCTGTACAGCAGCAGCCCCAGGGTGCTGAGC
AACAACAGTGATGCCAACCTGGAGCTGATCAACACCTGGGTGGCCAAGAACACCAACAACAAG
ATCAGCAGGCTGCTGGACAGCCTGCCCAGTGACACCAGGCTGGTGCTGCTGAATGCCATCTAC
CTGAGTGCCAAGTGGAAGACCACCTTTGACCCCAAGAAGACCAGGATGGAGCCCTTCCACTTC
AAGAACAGTGTGATCAAGGTGCCCATGATGAACAGCAAGAAGTACCCTGTGGCCCACTTCATT
GACCAGACCCTGAAGGCCAAGGTGGGCCAGCTTCAGCTTAGCCACAACCTGAGCCTGGTGATC
CTGGTGCCCCAGAACCTGAAGCACAGGCTGGAGGACATGGAGCAGGCCCTGAGCCCCAGTGTG
TTCAAGGCCATCATGGAGAAGCTGGAGATGAGCAAGTTCCAGCCCACCCTGCTGACCCTGCCC
AGGATCAAGGTGACCACCAGCCAGGACATGCTGAGCATCATGGAGAAGCTGGAGTTCTTTGAC
TTCAGCTATGACCTGAACCTGTGTGGCCTGACAGAGGACCCTGACCTGCAAGTCAGTGCCATG
CAGCACCAGACAGTGCTGGAGCTGACAGAGACAGGAGTGGAGGCTGCTGCTGCCAGTGCCATC
AGTGTGGCCAGGACCCTGCTGGTGTTTGAGGTGCAGCAGCCCTTCCTGTTTGTGCTGTGGGAC
CAGCAGCACAAGTTCCCTGTGTTCATGGGCAGGGTGTATGACCCCAGGGCCTAG
(SEQ ID NO: 4)
HA04
ATGGCCTCCAGGCTGACCCTGCTGACCCTCCTGCTGCTGCTGCTGGCTGGGGATAGAGCCTCC
TCAAATCCAAATGCTACCAGCTCCAGCTCCCAGGATCCAGAGAGTTTGCAAGACAGAGGAGAA
GGCAAGGTGGCAACAACAGTTATCTCCAAGATGCTATTTGTTGAACCCATCCTGGAGGTTTCC
AGCTTGCCTACAACCAACTCAACAACCAATTCAGCCACCAAAATAACAGCTAATACCACTGAT
GAACCCACCACACAACCCACCACAGAGCCCACCACCCAACCCACCATCCAACCCACCCAACCA
ACTACCCAGCTCCCAACAGATTCTCCTACCCAGCCCACTACTGGCTCCTTCTGCCCAGGACCT
GTTACTCTCTGCTCTGACTTGGAGAGTCATTCAACAGAGGCTGTGTTGGGAGATGCTTTGGTA
GATTTCTCCCTGAAGCTCTACCATGCCTTCTCAGCAATGAAGAAGGTGGAGACCAACATGGCC
TTTTCCCCATTCAGCATTGCCAGCCTCCTTACCCAGGTCCTGCTGGGGGCTGGAGAGAACACC
AAAACAAACCTGGAGAGCATCCTCTCTTACCCCAAGGACTTCACCTGTGTCCATCAGGCCCTG
AAGGGCTTCACTACCAAAGGTGTCACCTCAGTCTCTCAGATCTTCCATAGCCCAGACCTGGCC
ATAAGGGACACCTTTGTGAATGCCTCTAGGACCCTGTACAGCAGCAGCCCCAGAGTCCTAAGC
AACAACAGTGATGCCAACTTGGAGCTCATCAACACCTGGGTGGCCAAGAACACCAACAACAAG
ATCAGCAGGCTGCTAGACAGTCTGCCCTCTGATACCAGGCTTGTCCTCCTCAATGCTATCTAC
CTGAGTGCCAAGTGGAAGACAACATTTGATCCCAAGAAAACCAGAATGGAACCCTTTCACTTC
AAAAACTCAGTTATAAAAGTGCCCATGATGAATAGCAAGAAGTACCCTGTGGCCCATTTCATT
GACCAAACTTTGAAGGCCAAAGTGGGCCAGCTCCAGCTGTCCCACAATCTGAGTTTGGTGATC
CTGGTACCCCAGAACCTGAAACATAGACTTGAAGACATGGAACAGGCTCTCAGCCCTTCTGTT
TTCAAGGCCATCATGGAGAAACTGGAGATGTCCAAGTTCCAGCCCACTCTCCTAACACTACCC
AGGATCAAAGTGACTACCAGCCAGGATATGCTCTCAATCATGGAGAAATTGGAATTCTTTGAT
TTTTCTTATGACCTTAACCTGTGTGGCCTGACAGAGGACCCAGATCTTCAGGTTTCTGCTATG
CAGCATCAGACAGTGCTGGAACTGACAGAGACTGGAGTGGAGGCTGCTGCAGCCTCAGCCATC
TCTGTGGCCAGGACCCTGCTGGTCTTTGAAGTGCAGCAGCCCTTCCTCTTTGTGCTCTGGGAC
CAGCAGCACAAGTTCCCTGTCTTCATGGGCAGAGTATATGACCCCAGGGCCTAG
(SEQ ID NO: 5)
HA05
ATGGCCTCCAGGCTGACCCTGCTGACCCTCCTGCTGCTGCTGCTGGCTGGGGACAGAGCCTCC
TCAAATCCAAATGCTACCAGCTCCAGCTCCCAGGACCCAGAGAGCCTCCAAGACAGAGGAGAA
GGCAAGGTGGCAACAACAGTCATCTCCAAGATGCTCTTTGTGGAACCCATCCTGGAGGTCTCC
AGCCTCCCTACAACCAACTCAACAACCAATTCAGCCACCAAGATAACAGCTAATACCACTGAT
GAACCCACCACACAACCCACCACAGAGCCCACCACCCAACCCACCATCCAACCCACCCAACCA
ACTACCCAGCTCCCAACAGACTCTCCTACCCAGCCCACTACTGGCTCCTTCTGCCCAGGACCT
GTCACTCTCTGCTCTGACCTGGAGAGCCACTCAACAGAGGCTGTGCTGGGAGATGCTCTGGTG
GACTTCTCCCTGAAGCTCTACCATGCCTTCTCAGCAATGAAGAAAGTGGAGACCAACATGGCC
TTTTCCCCATTCAGCATTGCCAGCCTCCTGACCCAGGTCCTGCTGGGGGCTGGAGAGAACACC
AAGACAAACCTGGAGAGCATCCTCTCTTACCCCAAGGACTTCACCTGTGTCCATCAGGCCCTG
AAGGGCTTCACTACCAAGGGTGTCACCTCAGTCTCTCAGATCTTCCACAGCCCAGACCTGGCC
ATAAGGGACACCTTTGTGAATGCCTCTAGGACCCTGTACAGCAGCAGCCCCAGAGTCCTCAGC
AACAACAGTGATGCCAACCTGGAGCTCATCAACACCTGGGTGGCCAAGAACACCAACAACAAG
ATCAGCAGGCTGCTGGACAGCCTGCCCTCTGACACCAGGCTGGTCCTCCTCAATGCTATCTAC
CTGAGTGCCAAGTGGAAGACAACATTTGATCCCAAGAAAACCAGAATGGAACCCTTTCACTTC
AAGAACTCAGTCATAAAGGTGCCCATGATGAATAGCAAGAAATACCCTGTGGCCCACTTCATT
GACCAAACTCTCAAGGCCAAGGTGGGCCAGCTCCAGCTGTCCCACAATCTGAGCCTGGTGATC
CTGGTGCCCCAGAACCTGAAGCACAGACTGGAAGACATGGAACAGGCTCTCAGCCCTTCTGTC
TTCAAGGCCATCATGGAGAAGCTGGAGATGTCCAAGTTCCAGCCCACTCTCCTCACACTCCCC
AGGATCAAGGTCACTACCAGCCAGGACATGCTCTCAATCATGGAGAAGCTGGAATTCTTTGAC
TTTTCTTATGACCTGAACCTGTGTGGCCTGACAGAGGACCCAGATCTCCAGGTCTCTGCTATG
CAGCACCAGACAGTGCTGGAACTGACAGAGACTGGAGTGGAGGCTGCTGCAGCCTCAGCCATC
TCTGTGGCCAGGACCCTGCTGGTCTTTGAAGTGCAGCAGCCCTTCCTCTTTGTGCTCTGGGAC
CAGCAGCACAAGTTCCCTGTCTTCATGGGCAGAGTGTATGACCCCAGGGCCTAG
(SEQ ID NO: 6)
HA06
ATGGCCTCCAGGCTGACCCTGCTGACCCTCCTGCTGCTGCTGCTGGCTGGGGACAGGGCCTCC
TCAAACCCCAATGCCACCAGCTCCAGCTCCCAGGACCCAGAGAGCCTCCAGGACAGGGGGGAA
GGCAAGGTGGCTACCACAGTCATCTCCAAGATGCTCTTTGTGGAGCCCATCCTGGAGGTCTCC
AGCCTCCCCACCACCAACTCAACCACCAACTCAGCCACCAAGATCACAGCCAACACCACTGAT
GAGCCCACCACACAGCCCACCACAGAGCCCACCACCCAGCCCACCATCCAGCCCACCCAGCCC
ACCACCCAGCTCCCCACAGACTCCCCCACCCAGCCCACCACTGGCTCCTTCTGCCCAGGCCCT
GTCACCCTCTGCTCTGACCTGGAGAGCCACTCAACAGAGGCTGTGCTGGGGGATGCCCTGGTG
GACTTCTCCCTGAAGCTCTACCATGCCTTCTCAGCCATGAAGAAGGTGGAGACCAACATGGCC
TTCTCCCCCTTCAGCATTGCCAGCCTCCTGACCCAGGTCCTGCTGGGGGCTGGGGAGAACACC
AAGACCAACCTGGAGAGCATCCTCTCCTACCCCAAGGACTTCACCTGTGTCCATCAGGCCCTG
AAGGGCTTCACCACCAAGGGAGTCACCTCAGTCTCCCAGATCTTCCACAGCCCAGACCTGGCC
ATCAGGGACACCTTTGTGAATGCCTCCAGGACCCTGTACAGCAGCAGCCCCAGGGTCCTCAGC
AACAACAGTGATGCCAACCTGGAGCTCATCAACACCTGGGTGGCCAAGAACACCAACAACAAG
ATCAGCAGGCTGCTGGACAGCCTGCCCTCTGACACCAGGCTGGTCCTCCTCAATGCCATCTAC
CTGAGTGCCAAGTGGAAGACCACCTTTGATCCCAAGAAGACCAGGATGGAACCCTTCCACTTC
AAGAACTCAGTCATCAAGGTGCCCATGATGAACAGCAAGAAGTACCCTGTGGCCCACTTCATT
GACCAGACCCTCAAGGCCAAGGTGGGCCAGCTCCAGCTGTCCCACAACCTGAGCCTGGTGATC
CTGGTGCCCCAGAACCTGAAGCACAGGCTGGAGGACATGGAACAGGCCCTCAGCCCCTCTGTC
TTCAAGGCCATCATGGAGAAGCTGGAGATGTCCAAGTTCCAGCCCACCCTCCTCACCCTCCCC
AGGATCAAGGTGACCACCAGCCAGGACATGCTCTCAATCATGGAGAAGCTGGAGTTCTTTGAC
TTCTCCTATGACCTGAACCTGTGTGGCCTGACAGAGGACCCAGATCTCCAGGTCTCTGCCATG
CAGCACCAGACAGTGCTGGAACTGACAGAGACTGGAGTGGAGGCTGCTGCAGCCTCAGCCATC
TCTGTGGCCAGGACCCTGCTGGTCTTTGAAGTGCAGCAGCCCTTCCTCTTTGTGCTCTGGGAC
CAGCAGCACAAGTTCCCTGTCTTCATGGGCAGGGTGTATGACCCCAGGGCCTAG
(SEQ ID NO: 7)
Exemplary codon-optimized SERPING1 sequences
HA11
ATGGCATCAAGGCTGACCCTTCTCACCCTGCTCCTGCTGCTTCTGGCTGGGGACAGAGCCTCC
AGCAACCCCAATGCCACCTCCAGCAGCTCTCAAGATCCAGAGTCCCTGCAAGACCGGGGAGAA
GGCAAGGTGGCTACCACAGTCATCTCCAAGATGCTCTTTGTGGAACCCATCCTGGAAGTGTCC
TCCCTCCCCACCACTAACTCCACCACAAACTCTGCCACTAAGATCACAGCCAACACTACAGAT
GAGCCAACTACCCAGCCTACAACAGAGCCTACCACCCAACCAACCATCCAGCCAACCCAGCCC
ACCACTCAGCTGCCCACAGACAGCCCAACACAGCCCACAACTGGCTCCTTCTGCCCTGGCCCT
GTGACCCTCTGCAGTGACTTGGAAAGCCACAGCACCGAGGCTGTGCTGGGAGATGCTCTGGTG
GACTTTTCTCTCAAGCTGTACCATGCTTTCTCAGCAATGAAGAAAGTAGAGACCAACATGGCC
TTCAGCCCCTTTTCCATTGCCAGCCTCCTCACTCAAGTGCTCCTGGGGGCTGGAGAGAATACC
AAAACCAACCTGGAGAGCATCCTCTCCTACCCCAAGGACTTCACCTGTGTGCACCAAGCCCTG
AAAGGCTTCACAACCAAGGGGGTGACTTCTGTCTCCCAGATCTTCCACTCTCCAGACCTGGCC
ATCAGAGACACCTTCGTGAATGCAAGTAGGACCCTCTACAGCTCCTCCCCAAGGGTGCTGTCC
AACAATTCTGATGCCAATCTGGAGCTCATCAACACCTGGGTGGCCAAGAACACAAACAACAAG
ATTTCTAGGCTCCTGGACTCCCTGCCTTCTGACACAAGACTGGTTCTGCTGAATGCTATCTAC
CTGTCTGCAAAGTGGAAGACCACCTTTGACCCCAAAAAGACAAGGATGGAGCCCTTCCATTTT
AAGAATTCAGTGATCAAGGTGCCCATGATGAACAGCAAGAAGTACCCTGTTGCCCACTTCATT
GACCAAACACTGAAGGCCAAAGTGGGCCAGCTCCAGCTTTCCCACAACCTCAGCCTGGTGATT
CTGGTCCCCCAGAACTTGAAGCACCGGCTGGAGGACATGGAGCAAGCACTGAGCCCCAGTGTC
TTCAAGGCAATCATGGAAAAACTGGAGATGTCCAAATTCCAGCCCACTCTGTTGACCTTGCCC
AGAATCAAAGTCACCACCAGCCAAGATATGCTGAGTATCATGGAGAAGCTTGAGTTCTTTGAT
TTCTCCTATGATCTGAACCTGTGTGGCCTGACAGAAGACCCTGACCTCCAAGTCAGTGCCATG
CAGCACCAGACAGTGTTGGAGCTGACTGAGACTGGTGTGGAGGCAGCAGCCGCCTCAGCCATC
TCTGTGGCCAGAACCCTGCTGGTGTTTGAGGTGCAGCAGCCTTTCCTGTTTGTCCTGTGGGAC
CAGCAACACAAGTTCCCAGTGTTCATGGGCAGAGTGTATGACCCAAGGGCCTGA
(SEQ ID NO: 8)
HA12
ATGGCAAGCAGACTGACCTTGCTGACACTGCTCCTTCTCCTGCTGGCTGGAGACCGGGCCAGC
AGCAACCCCAATGCCACTTCTTCCTCCAGTCAAGACCCAGAGTCCCTGCAAGACAGAGGAGAA
GGCAAGGTTGCTACCACTGTGATCTCCAAGATGCTGTTCGTGGAGCCCATCCTGGAAGTGTCC
AGCCTGCCCACCACTAACTCCACCACAAACAGTGCCACCAAAATCACAGCCAACACTACAGAT
GAGCCAACCACCCAACCAACCACAGAACCCACAACACAGCCTACCATCCAGCCTACTCAGCCC
ACCACCCAGCTTCCTACAGACTCCCCCACCCAGCCAACAACTGGCTCCTTCTGCCCTGGCCCT
GTGACTCTGTGCTCTGACCTGGAGAGCCACAGCACCGAGGCTGTGCTGGGAGATGCTCTGGTG
GACTTCTCCCTGAAACTGTACCACGCCTTTTCAGCCATGAAGAAAGTAGAGACCAACATGGCC
TTCAGCCCCTTTAGCATTGCCTCCCTCCTCACTCAAGTTCTGCTGGGGGCTGGGGAGAACACA
AAGACCAACCTGGAAAGCATCCTCTCCTACCCCAAGGACTTCACCTGTGTGCACCAAGCACTC
AAGGGCTTCACAACCAAGGGGGTGACCAGTGTCTCCCAGATCTTCCATTCCCCAGACCTGGCC
ATCAGAGACACATTTGTGAATGCATCCAGAACCCTCTACAGCTCCTCTCCAAGGGTGCTGTCC
AACAACTCAGATGCCAATCTTGAGCTGATCAACACCTGGGTGGCCAAAAATACCAATAACAAG
ATCTCAAGGCTGCTGGACAGCCTCCCCAGTGACACCAGACTTGTGCTCCTGAATGCTATCTAC
CTGTCTGCAAAGTGGAAGACTACCTTTGATCCCAAAAAGACAAGGATGGAGCCTTTCCACTTC
AAGAACTCTGTCATCAAAGTGCCCATGATGAACAGCAAGAAGTACCCTGTTGCCCACTTTATT
GATCAGACCCTGAAGGCCAAGGTGGGCCAGCTCCAGCTGTCCCACAACCTTTCTCTGGTGATT
CTGGTCCCCCAGAACCTGAAGCACAGATTGGAAGACATGGAGCAAGCCCTGAGCCCTTCAGTG
TTTAAGGCAATCATGGAGAAGCTGGAGATGTCCAAATTCCAGCCCACTCTCCTCACCCTGCCT
AGGATCAAGGTCACCACCAGCCAAGATATGCTCTCTATCATGGAAAAGTTGGAGTTCTTTGAC
TTTTCCTATGATCTGAACCTCTGTGGCCTGACTGAGGACCCTGACCTCCAAGTGTCAGCAATG
CAGCACCAGACAGTGCTGGAGCTCACAGAGACTGGGGTGGAAGCCGCAGCAGCCTCTGCCATC
TCTGTGGCTAGGACCCTTCTGGTCTTTGAGGTGCAGCAGCCCTTCCTGTTTGTCCTGTGGGAC
CAGCAACACAAGTTCCCAGTGTTCATGGGCAGAGTGTATGACCCCAGAGCCTGA
(SEQ ID NO: 9)
HA13
ATGGCATCTAGGCTGACCTTGCTCACTCTGCTGCTTCTCCTCCTGGCTGGAGACCGGGCCTCC
AGCAACCCCAATGCTACCTCCTCCAGTTCCCAAGATCCAGAGTCCCTGCAAGACAGAGGAGAA
GGCAAGGTGGCAACCACTGTCATCAGCAAGATGCTGTTTGTGGAACCCATCCTGGAAGTGTCC
AGCCTCCCCACAACCAACTCCACCACAAATTCAGCCACCAAGATCACTGCCAACACAACTGAT
GAGCCCACCACCCAACCAACCACAGAGCCAACTACCCAGCCCACCATCCAGCCCACTCAGCCT
ACCACCCAGCTTCCTACTGACAGCCCAACACAGCCAACCACTGGCTCCTTCTGCCCTGGCCCT
GTGACCCTGTGCTCTGACTTGGAAAGCCACAGCACTGAAGCTGTGCTGGGAGATGCTCTGGTG
GACTTTTCCCTCAAGCTGTACCACGCCTTCTCTGCAATGAAGAAGGTTGAGACCAACATGGCC
TTTTCTCCCTTCAGCATTGCCAGCCTGCTGACTCAAGTGCTGCTGGGGGCTGGGGAGAACACC
AAAACAAACCTGGAGAGCATCCTCTCCTACCCCAAAGATTTCACCTGTGTGCACCAAGCCCTG
AAAGGCTTCACAACAAAGGGGGTCACCTCTGTCTCCCAGATCTTCCATTCCCCTGACCTGGCC
ATCAGAGACACCTTCGTGAATGCCAGCAGAACCCTCTACAGCAGCTCCCCAAGGGTGCTCTCC
AACAATTCTGATGCCAATCTGGAGCTCATCAACACCTGGGTGGCCAAAAACACTAACAACAAG
ATTTCCAGACTCCTGGACTCCCTGCCCAGTGACACAAGGTTGGTCCTCCTGAATGCAATCTAC
CTGTCTGCTAAGTGGAAGACCACCTTTGATCCCAAGAAAACCAGAATGGAACCATTCCACTTT
AAGAACTCAGTGATCAAGGTCCCCATGATGAACAGCAAAAAGTACCCTGTTGCCCACTTCATT
GATCAGACCCTGAAGGCCAAGGTGGGCCAGCTCCAGCTGTCCCACAACCTCTCTCTGGTCATT
CTGGTGCCCCAGAACTTGAAGCACCGGCTGGAAGATATGGAGCAAGCACTGAGCCCCTCTGTG
TTCAAGGCAATTATGGAGAAGCTTGAGATGTCCAAGTTCCAGCCTACACTGCTCACCCTGCCT
AGGATCAAAGTGACTACCAGCCAAGACATGCTCTCAATCATGGAGAAACTGGAGTTCTTTGAC
TTCTCCTATGATCTGAACCTGTGTGGCCTGACTGAGGACCCAGACCTCCAAGTCAGTGCCATG
CAGCACCAGACAGTGTTGGAGCTGACAGAAACTGGGGTGGAGGCAGCAGCCGCCTCTGCCATC
AGTGTGGCTAGGACCCTGTTGGTGTTTGAGGTGCAGCAGCCCTTTCTCTTTGTCCTGTGGGAC
CAGCAACACAAGTTTCCTGTCTTCATGGGCAGAGTGTATGACCCCAGAGCCTGA
(SEQ ID NO: 10)
HA14
ATGGCCAGCAGACTCACCCTCCTCACTCTGCTCCTGCTTCTGCTGGCTGGGGACCGGGCCTCT
AGCAACCCCAATGCCACCAGCTCCTCCAGCCAAGATCCAGAATCTCTGCAAGACAGAGGAGAA
GGCAAGGTTGCCACTACTGTGATCAGCAAGATGCTCTTTGTGGAGCCCATCCTGGAAGTGAGC
AGCCTCCCCACTACCAACAGCACCACTAACTCAGCTACCAAGATCACAGCCAATACCACAGAT
GAACCCACCACACAGCCTACAACAGAACCTACCACTCAACCCACCATCCAGCCTACTCAGCCC
ACCACCCAGCTGCCCACAGACTCCCCAACCCAGCCAACAACTGGCTCCTTCTGCCCTGGCCCT
GTCACACTCTGCAGTGACTTGGAGAGCCACAGCACTGAGGCTGTGCTGGGAGATGCCCTGGTG
GACTTTTCCCTCAAGCTCTACCACGCCTTCTCAGCAATGAAGAAAGTAGAGACCAACATGGCA
TTCTCTCCTTTCAGCATTGCCTCCCTGCTGACCCAAGTCCTCCTGGGGGCTGGAGAGAACACT
AAAACCAATCTTGAGTCCATCCTCTCCTACCCCAAGGACTTCACCTGTGTCCACCAAGCACTG
AAAGGCTTCACAACCAAAGGAGTGACCTCTGTGTCCCAGATCTTCCATTCCCCTGATCTGGCC
ATCAGAGACACCTTCGTGAATGCTTCCAGAACCTTGTACAGCTCTAGCCCAAGGGTGCTGTCC
AACAATTCTGATGCTAATCTGGAGCTGATCAACACCTGGGTGGCCAAAAACACAAACAACAAG
ATTTCTAGGCTCCTGGACAGCCTGCCTTCTGACACAAGGCTGGTTCTGCTGAATGCAATCTAC
CTGTCTGCAAAGTGGAAGACCACCTTTGACCCCAAAAAGACAAGGATGGAGCCATTCCACTTC
AAGAACAGTGTCATCAAAGTGCCCATGATGAACTCCAAGAAGTACCCAGTGGCCCATTTCATT
GATCAGACCCTGAAGGCCAAGGTGGGCCAGCTCCAGCTTTCCCACAACCTCTCACTGGTGATT
CTGGTCCCCCAGAACCTGAAGCACCGGCTGGAAGATATGGAGCAAGCCCTCAGCCCCAGTGTG
TTTAAGGCTATCATGGAGAAGCTGGAGATGTCCAAATTCCAGCCCACTCTCCTGACACTGCCA
AGGATCAAGGTCACCACCTCCCAAGACATGCTGTCAATCATGGAAAAACTGGAGTTCTTTGAT
TTCTCCTATGACCTGAACCTGTGTGGCCTGACTGAGGACCCTGACCTCCAAGTGTCTGCCATG
CAGCACCAGACAGTGCTGGAGCTCACAGAGACTGGGGTGGAAGCCGCTGCTGCCTCAGCCATC
TCAGTGGCTAGGACCCTGCTGGTCTTTGAGGTGCAGCAGCCCTTCCTGTTTGTCCTGTGGGAC
CAGCAACACAAGTTCCCTGTGTTCATGGGCAGAGTGTATGATCCCAGAGCCTGA
(SEQ ID NO: 11)
HA15
ATGGCATCTAGGCTGACACTGCTCACCTTGCTCCTGCTGCTTCTGGCTGGGGACAGAGCTTCC
TCCAACCCCAACGCCACCTCCAGCTCTTCCCAAGACCCTGAGTCTCTGCAAGATAGGGGGGAG
GGCAAGGTGGCTACCACAGTCATCAGCAAGATGCTCTTTGTGGAACCCATCCTGGAAGTGAGC
AGCCTCCCCACTACCAATAGCACCACCAACAGTGCCACAAAGATCACAGCCAACACTACAGAT
GAGCCAACCACTCAGCCCACAACAGAGCCTACTACCCAGCCCACCATCCAGCCCACACAGCCT
ACCACCCAGCTTCCTACAGACAGCCCCACTCAACCCACCACTGGCTCCTTCTGCCCTGGCCCT
GTGACCCTCTGCAGTGACTTGGAGAGCCACAGCACTGAGGCTGTGCTGGGAGATGCTCTGGTG
GACTTTTCCCTCAAGCTCTACCACGCCTTCTCTGCAATGAAGAAGGTTGAGACCAACATGGCC
TTTAGCCCATTCAGCATTGCCTCCTTGCTGACCCAAGTCCTGCTGGGGGCTGGAGAGAACACC
AAAACAAATCTTGAGTCCATCCTCTCCTACCCCAAAGACTTCACCTGTGTGCACCAAGCACTG
AAAGGCTTCACAACCAAGGGGGTGACTTCTGTGTCCCAGATCTTCCACTCTCCTGACCTGGCC
ATCAGAGACACCTTTGTCAATGCCAGCAGAACCCTTTACAGCTCCTCCCCAAGAGTCCTCAGC
AACAATTCTGATGCCAATCTGGAGCTGATCAACACCTGGGTGGCAAAAAACACAAACAACAAG
ATTTCCAGACTCCTGGACTCCCTGCCTTCTGACACAAGGCTGGTTCTGCTGAATGCTATCTAC
CTGTCTGCCAAGTGGAAGACCACATTTGACCCCAAGAAAACCAGAATGGAGCCCTTCCATTTC
AAGAACTCAGTGATCAAGGTCCCCATGATGAACAGCAAAAAGTACCCTGTTGCCCACTTCATT
GATCAGACCCTGAAGGCCAAAGTGGGCCAGCTCCAGCTGTCCCACAACCTGAGCCTGGTCATT
CTGGTGCCCCAGAACCTGAAGCACAGACTGGAAGATATGGAGCAAGCCCTGAGCCCTTCAGTC
TTTAAGGCAATCATGGAGAAGCTGGAGATGTCCAAGTTCCAGCCAACTCTCCTCACTCTGCCC
AGAATCAAAGTCACCACCAGCCAAGACATGCTGAGTATCATGGAAAAACTGGAGTTCTTTGAT
TTCTCCTATGACCTGAACCTCTGTGGCCTGACTGAGGACCCAGACCTCCAAGTGTCTGCCATG
CAGCACCAGACAGTGCTGGAGCTCACAGAAACTGGTGTGGAGGCCGCTGCTGCCTCAGCCATC
AGTGTGGCCCGCACCCTGTTGGTGTTTGAGGTGCAGCAGCCTTTCCTGTTCGTGCTGTGGGAC
CAGCAACACAAGTTTCCAGTGTTCATGGGCAGAGTGTATGATCCAAGGGCCTGA
(SEQ ID NO: 12)
HA16
ATGGCCTCTAGGCTCACCCTGCTGACACTGCTGCTCCTCCTGCTGGCTGGAGACCGGGCCAGC
AGCAACCCCAATGCTACCAGCTCCTCCAGTCAAGACCCTGAGTCCCTGCAAGACAGAGGAGAA
GGCAAGGTTGCCACTACAGTCATCTCCAAGATGCTGTTTGTAGAGCCCATCCTGGAAGTGTCC
AGCCTGCCTACCACCAATAGCACCACCAACAGTGCCACCAAGATCACAGCCAATACCACAGAT
GAGCCAACCACCCAGCCTACAACAGAGCCTACCACTCAACCCACCATCCAGCCAACACAGCCT
ACTACCCAGCTGCCCACAGACAGCCCAACTCAGCCCACCACTGGCTCCTTCTGCCCTGGCCCT
GTCACACTCTGCAGTGACCTGGAAAGCCACAGCACTGAGGCTGTGCTGGGAGATGCTCTGGTG
GACTTTTCTCTGAAACTGTACCACGCCTTCTCTGCAATGAAGAAAGTGGAGACCAACATGGCA
TTCTCCCCATTCAGCATTGCCTCCTTGCTGACCCAAGTGCTCCTGGGGGCTGGGGAGAACACA
AAAACAAACCTGGAGAGCATCCTCTCCTACCCCAAGGACTTCACCTGTGTGCACCAAGCCCTC
AAGGGCTTCACAACCAAAGGAGTGACCTCTGTGAGCCAGATCTTCCATTCCCCTGACCTGGCC
ATTAGGGACACCTTCGTGAATGCAAGCCGGACCCTCTACAGCAGCTCTCCAAGAGTCCTGTCC
AACAATTCTGATGCCAACCTAGAGCTGATCAACACCTGGGTGGCAAAGAACACTAACAACAAG
ATTTCCAGACTCCTGGACTCCCTCCCATCTGACACAAGGCTGGTCCTCCTGAATGCCATCTAC
CTGTCTGCTAAGTGGAAGACCACATTTGACCCCAAAAAGACAAGGATGGAACCCTTCCACTTC
AAAAACTCAGTGATCAAAGTCCCCATGATGAACAGCAAGAAGTACCCTGTGGCCCATTTCATT
GATCAGACCCTGAAGGCCAAGGTGGGCCAGCTCCAGCTTTCCCACAACCTCAGCCTTGTGATT
CTGGTGCCCCAGAACTTGAAGCACAGACTGGAGGACATGGAGCAAGCCCTGAGCCCCAGTGTC
TTCAAGGCAATCATGGAGAAACTTGAGATGTCCAAATTCCAGCCCACTCTGCTCACTCTCCCC
AGAATCAAGGTCACCACCTCCCAAGATATGCTCTCAATTATGGAGAAGCTGGAGTTCTTTGAT
TTTTCCTATGATCTGAACCTGTGTGGCCTGACTGAAGATCCAGACCTCCAAGTCTCTGCCATG
CAGCACCAGACAGTGTTGGAGCTCACAGAAACTGGGGTGGAAGCCGCTGCTGCCTCAGCCATC
AGTGTGGCTAGGACCCTGTTGGTGTTTGAGGTGCAGCAGCCCTTTCTCTTTGTGCTGTGGGAC
CAGCAACACAAGTTCCCAGTGTTCATGGGCAGAGTGTATGACCCAAGGGCCCTGA
(SEQ ID NO: 13)
HA17
ATGGCATCCCGCCTGACCCTCCTCACCCTGCTTCTGCTCCTGCTGGCTGGAGATAGGGCCTCC
AGCAACCCCAATGCAACCAGCAGCTCCAGCCAAGACCCTGAGTCCCTGCAAGACAGAGGAGAA
GGCAAGGTTGCCACCACTGTGATTTCCAAGATGCTGTTTGTGGAACCCATCCTGGAAGTGAGC
AGCCTCCCAACAACCAACAGCACCACCAATAGTGCCACAAAAATTACAGCCAATACCACAGAT
GAGCCTACCACTCAGCCTACAACAGAACCCACCACCCAGCCTACCATCCAGCCGACCCAGCCA
ACCACACAGCTCCCCACTGACTCCCCCACACAGCCCACCACTGGCTCCTTCTGCCCTGGACCT
GTGACTCTGTGCTCTGACCTGGAATCTCACTCCACTGAGGCTGTGCTGGGGGATGCTCTGGTG
GATTTTTCCCTCAAGCTCTACCATGCCTTCTCTGCCATGAAGAAGGTGGAGACCAACATGGCC
TTTTCTCCCTTTTCCATTGCCAGCCTCCTGACCCAAGTGCTCCTGGGAGCTGGGGAGAACACC
AAGACCAACCTTGAGAGCATTCTGTCCTACCCCAAGGACTTTACCTGTGTGCACCAAGCCCTG
AAAGGCTTCACCACCAAAGGGGTGACCTCTGTCTCCCAGATCTTCCACAGCCCTGATCTGGCC
ATTAGGGACACCTTTGTCAATGCTTCCAGAACTCTCTATTCTTCCTCCCCACGGGTCCTCTCC
AACAATTCAGATGCCAACCTGGAGCTGATCAACACCTGGGTGGCCAAAAACACAAACAACAAG
ATCTCAAGGCTCCTGGACAGCCTCCCCAGTGACACACGGCTGGTGCTCCTGAATGCCATCTAC
CTGTCTGCCAAGTGGAAGACCACTTTTGACCCAAAAAAGACAAGGATGGAGCCCTTCCATTTC
AAGAACAGTGTGATCAAGGTCCCCATGATGAATAGCAAGAAATACCCTGTTGCCCACTTCATT
GATCAGACCCTGAAGGCCAAAGTGGGCCAGCTGCAGTTGTCCCACAACCTCAGCCTGGTCATA
CTGGTGCCCCAGAACCTGAAGCATAGGCTGGAAGACATGGAACAAGCACTGAGCCCCTCAGTG
TTCAAAGCTATCATGGAGAAGCTGGAGATGTCCAAATTCCAGCCCACCCTGTTGACCCTGCCT
AGGATCAAAGTCACCACCTCCCAAGATATGCTCTCAATAATGGAGAAACTGGAGTTCTTTGAT
TTCTCCTATGATCTCAATCTGTGTGGCCTGACTGAAGATCCAGACCTCCAAGTCTCAGCAATG
CAGCACCAGACAGTCCTGGAGCTCACAGAGACTGGTGTGGAGGCGGCAGCAGCCTCAGCCATC
TCTGTGGCCAGAACCCTGCTGGTGTTTGAGGTGCAGCAGCCCTTCCTGTTTGTTCTGTGGGAC
CAGCAGCATAAGTTTCCTGTCTTCATGGGTAGGGTGTATGACCCCAGAGCCTGA
(SEQ ID NO: 14)
HA18
ATGGCCAGCAGACTCACCCTGCTGACCCTGCTCCTCCTGCTCCTGGCTGGGGACAGAGCCTCC
TCCAACCCCAATGCAACCTCCTCCAGCTCCCAAGACCCTGAGTCCCTGCAAGATAGGGGAGAA
GGCAAGGTTGCCACTACTGTCATAAGCAAGATGCTGTTTGTTGAACCCATCTTGGAGGTGTCC
AGTCTCCCCACCACAAACAGCACCACCAACTCTGCCACCAAGATCACTGCCAATACCACAGAT
GAACCCACCACTCAGCCCACAACTGAGCCCACCACCCAGCCAACCATACAGCCTACACAGCCA
ACAACCCAGCTGCCCACTGACTCCCCAACCCAGCCTACCACTGGCAGCTTCTGCCCTGGTCCT
GTCACCCTCTGCAGTGACCTGGAAAGCCATAGCACAGAGGCTGTGCTGGGGGATGCTCTGGTG
GACTTCTCCCTCAAACTCTACCATGCTTTTTCTGCAATGAAGAAGGTGGAAACCAACATGGCC
TTCAGCCCCTTCTCCATTGCCAGCCTCTTGACCCAAGTGCTCCTGGGAGCTGGAGAGAACACT
AAGACTAATCTGGAGAGCATCCTCTCCTACCCCAAAGACTTCACCTGTGTGCACCAAGCCCTG
AAGGGCTTCACCACCAAAGGAGTGACCTCTGTCTCCCAGATCTTCCATTCCCCTGACCTGGCC
ATAAGGGACACCTTTGTGAATGCCTCTAGGACCCTGTACTCCAGCTCTCCTCGGGTCCTCAGC
AACAACTCAGATGCCAACCTGGAGCTCATCAACACCTGGGTGGCCAAAAATACCAATAACAAG
ATTTCCCGCCTCCTGGATTCCCTGCCCTCTGACACACGCCTGGTGCTCCTGAATGCCATCTAC
CTGAGTGCCAAGTGGAAGACCACCTTTGATCCCAAGAAAACCAGAATGGAGCCTTTCCACTTT
AAGAATTCAGTCATTAAAGTGCCCATGATGAATTCCAAGAAGTATCCTGTGGCTCACTTCATT
GACCAGACCCTCAAGGCCAAGGTAGGTCAGCTCCAGCTGAGCCACAACCTCAGCCTGGTGATC
CTGGTCCCCCAGAACCTGAAACATAGGCTGGAGGACATGGAACAAGCACTGTCCCCATCAGTC
TTCAAGGCTATCATGGAGAAGCTGGAGATGAGCAAATTCCAGCCCACACTGCTGACCTTGCCT
AGGATCAAGGTCACCACCAGCCAAGATATGCTCTCAATTATGGAGAAACTGGAATTCTTTGAC
TTTTCCTATGACCTCAATCTGTGTGGCCTGACAGAAGATCCAGATCTCCAAGTCAGTGCCATG
CAGCACCAGACAGTGCTGGAGCTGACTGAAACTGGGGTGGAGGCAGCGGCAGCCTCAGCCATT
TCTGTGGCAAGGACCCTGCTGGTGTTTGAAGTGCAGCAGCCCTTTCTCTTTGTCCTGTGGGAT
CAGCAGCACAAGTTTCCAGTGTTCATGGGCCGGGTGTATGATCCAAGGGCCTGA
(SEQ ID NO: 15)
HA19
ATGGCCTCCCGGCTGACCCTTCTCACCCTGCTCCTGCTGCTTCTGGCTGGGGACCGGGCCAGC
AGCAACCCCAATGCTACCAGCTCTTCATCACAAGACCCTGAGAGCCTGCAAGACAGAGGAGAA
GGCAAGGTTGCCACTACTGTGATCAGCAAGATGCTCTTCGTGGAGCCCATCCTGGAAGTGTCT
TCTCTGCCCACAACCAACTCCACCACAAATTCTGCAACCAAAATCACAGCCAATACCACTGAT
GAACCCACCACTCAACCCACCACAGAGCCAACAACACAGCCAACCATCCAGCCTACCCAGCCT
ACCACCCAGCTTCCTACAGACAGCCCAACTCAGCCCACAACTGGCTCCTTCTGCCCTGGGCCT
GTCACCCTGTGCTCTGACTTGGAAAGCCACAGCACCGAGGCTGTGCTGGGAGATGCTCTGGTG
GACTTTTCTTTGAAGCTCTACCACGCCTTCAGTGCCATGAAGAAAGTAGAGACCAACATGGCA
TTCAGCCCCTTTTCCATTGCTTCCCTGTTGACCCAAGTGCTCCTTGGAGCTGGAGAGAACACT
AAAACCAATCTTGAGTCCATCCTCTCCTACCCCAAAGACTTCACCTGTGTGCACCAAGCACTG
AAAGGCTTCACAACCAAGGGGGTGACCTCAGTGTCCCAGATCTTCCATTCCCCAGACCTGGCC
ATTAGGGACACATTTGTGAATGCAAGCAGAACTCTGTACAGCTCCAGCCCTAGGGTGCTGTCC
AACAATTCAGATGCCAACTTGGAGCTCATCAACACCTGGGTGGCCAAAAACACAAACAACAAG
ATCTCCAGACTCCTGGACTCCCTCCCCAGTGACACCAGACTGGTTCTGCTGAATGCCATCTAC
CTGTCAGCCAAGTGGAAGACCACCTTTGACCCCAAGAAGACAAGGATGGAACCATTCCACTTT
AAGAACAGTGTCATCAAAGTGCCCATGATGAACAGCAAAAAGTACCCTGTGGCCCACTTCATT
GATCAGACCCTCAAGGCAAAGGTGGGCCAGCTGCAGCTCTCCCACAACCTCAGCCTTGTGATT
CTGGTCCCCCAGAACCTGAAGCACAGACTGGAGGACATGGAACAAGCCCTGTCTCCTTCAGTC
TTCAAGGCCATCATGGAGAAGCTGGAGATGTCCAAGTTCCAGCCCACTCTCCTCACACTGCCT
AGAATCAAGGTCACTACCTCTCAAGATATGCTGAGCATCATGGAGAAACTGGAGTTCTTTGAT
TTCTCCTATGACCTCAACCTGTGTGGCCTGACAGAAGATCCAGATTTGCAAGTTTCTGCAATG
CAGCACCAGACAGTGCTAGAGCTGACTGAGACTGGTGTGGAAGCTGCTGCAGCCTCTGCTATC
TCTGTGGCAAGAACCTTGCTGGTGTTTGAGGTGCAGCAGCCCTTCCTGTTTGTCCTGTGGGAC
CAGCAACACAAGTTTCCAGTGTTCATGGGCAGAGTGTATGATCCCAGAGCCTGA
(SEQ ID NO: 16)
HA20
ATGGCATCCAGACTCACACTGCTCACTCTGCTGCTTCTGTTGCTGGCTGGAGACCGGGCCTCC
AGCAACCCCAACGCCACTTCTTCCTCCAGTCAAGATCCTGAGAGTCTCCAAGACAGAGGAGAA
GGCAAGGTTGCTACCACTGTGATTTCCAAGATGCTCTTTGTGGAGCCCATCTTGGAAGTGAGC
TCCCTGCCTACCACCAATTCCACCACAAATTCAGCCACCAAAATCACCGCCAACACTACAGAT
GAGCCAACCACCCAGCCCACGACAGAGCCTACAACTCAGCCCACCATCCAGCCTACTCAACCA
ACAACACAGCTCCCCACAGACAGCCCCACCCAGCCAACTACTGGCTCCTTCTGCCCTGGGCCC
GTGACCCTCTGCAGTGACCTTGAGAGCCACAGCACCGAGGCTGTGCTGGGAGATGCTCTGGTG
GACTTTTCTCTGAAGCTGTACCATGCTTTCTCTGCAATGAAGAAAGTAGAGACCAACATGGCC
TTCAGTCCCTTTTCCATTGCCAGCCTGCTGACCCAAGTGCTCCTGGGCGCTGGGGAGAACACA
AAAACCAACCTGGAGTCCATCCTCTCCTACCCCAAGGATTTCACCTGTGTGCACCAAGCCCTG
AAGGGCTTCACAACCAAGGGGGTCACCTCTGTGTCCCAGATCTTCCATTCCCCAGACCTGGCC
ATCAGAGACACCTTCGTGAATGCTTCCCGCACCCTCTACAGCAGCTCTCCTCGAGTGCTATCC
AACAATTCTGATGCCAATCTGGAGCTCATCAACACCTGGGTGGCAAAGAATACAAACAACAAG
ATCAGCCGGCTGCTGGACTCCCTCCCTTCTGACACAAGGCTGGTCCTCCTGAATGCCATCTAC
CTGTCTGCCAAGTGGAAGACCACATTTGATCCCAAAAAGACAAGAATGGAACCATTCCACTTT
AAGAACTCCGTGATCAAAGTCCCCATGATGAACAGCAAGAAGTACCCTGTGGCCCACTTCATT
GATCAGACCCTGAAAGCCAAAGTGGGCCAGCTGCAGCTTTCCCACAACCTGTCCTTGGTCATC
CTGGTGCCCCAGAACCTCAAGCACCGCCTGGAAGATATGGAACAAGCACTCAGCCCATCTGTC
TTCAAGGCCATTATGGAGAAACTGGAGATGTCCAAATTCCAGCCCACTCTCCTCACCCTGCCC
AGAATCAAGGTGACTACCAGCCAAGACATGCTGAGCATCATGGAGAAGCTCGAGTTCTTTGAC
TTCTCCTATGACCTCAACTTGTGTGGCCTGACTGAGGACCCTGATCTGCAAGTCAGTGCCATG
CAGCACCAGACAGTGTTGGAGCTGACAGAAACTGGTGTGGAAGCTGCTGCAGCCTCTGCTATG
TCAGTGGCTCGGACCTTGCTTGTGTTTGAGGTGCAGCAACCCTTCCTGTTTGTCCTGTGGGAC
CAGCAGCATAAGTTTCCAGTGTTCATGGGCCGGGTGTATGATCCAAGAGCCTGA
(SEQ ID NO: 17)
3XCRM8
GGGGGAGGCTGCTGGTGAATATTAACCAAGGTCACCCCAGTTATCGGAGGAGCAAACAGGGGC
TAAGTCCACCGGGGGAGGCTGCTGGTGAATATTAACCAAGGTCACCCCAGTTATCGGAGGAGC
AAACAGGGGCTAAGTCCACCGGGGGAGGCTGCTGGTGAATATTAACCAAGGTCACCCCAGTTA
TCGGAGGAGCAAACAGGGGCTAAGTCCAC
(SEQ ID NO: 18)
MVM intron
CTAAGGTAAGTTGGCGCCGTTTAAGGGATGGTTGGTTGGTGGGGTATTAATGTTTAATTACCT
TTTTTACAGGCCTG
(SEQ ID NO: 19)
SERPING1 exon1-intron1-partially exon 2
CTGATTTACAGGAACTCACACCAGCGATCAATCTTCCTTAATTTGTAACTGGGCAGTGTCCCG
GGCCAGCCAATAGCTAAGACTGCCCCCCCCGCACCCCACCCTCCCTGACCCTGGGGGACTCTC
TACTCAGTCTGCACTGGAGCTGCCTGGTGACCAGAAGTTTGGAGTAGGTTTGGTGCTGGGCAG
GGGTGGGGAGTAGGGTGGAAAGCATGGAGTGAAGAGGTCTAGGGAGGGGGTCTCCTCACCCCC
GCCTTCCTGCCCGCCTTGATCTCGGGGGTCTCTATAGGCTTGCTTCCACCTGGGACTTCTGCC
TCCTCCTACCCCAGCCCCTCCCGCCTCAGGCCTGTTGTGCTCAGCCCCCCAGGACCTCCCCTC
CCCCACGCCTCTGGCCTCATTGTTTGGTTAAAGCAGGACCCCCTCCCCCTCCCACCACCTCCC
CTCCGACTGAACAGATGGACAGAGACCCGGGCCCACGGGGAGAGGAAGGGCCAGCCGGTGCCG
GGAAAGGGAAGCGGTTTGGGGAAAACAAAACAGAGGGAGGAGCCAGGGAGAAGGTGGCCCCAG
GAGGGAGGAGGAGGGAATTCGCTAAGAGGGACTGGGGCCTGAGACGGAATGGGGGCGGGCCCC
GGGCGGGGTGGGGGCCCCTGGGCTCCCAGGGTGGGAGCTGGCTCCGAGGCTGGCTGGCTCCGC
AGGTCCGCTGACGTCGCCGCCCAG
(SEQ ID NO: 20)
BGHpA
CCTAGAGCTCGCTGATCAGCCTCGACTGTGCCTTCTAGTTGCCAGCCATCTGTTGTTTGCCCC
TCCCCCGTGCCTTCCTTGACCCTGGAAGGTGCCACTCCCACTGTCCTTTCCTAATAAAATGAG
GAAATTGCATCGCATTGTCTGAGTAGGTGTCATTCTATTCTGGGGGGTGGGGTGGGGCAGGAC
AGCAAGGGGGAGGATTGGGAAGACAATAGCAGGCATGCTGGGGAA
(SEQ ID NO: 21)
WPRE WT
AATCAACCTCTGGATTACAAAATTTGTGAAAGATTGACTGGTATTCTTAACTATGTTGCTCCT
TTTACGCTATGTGGATACGCTGCTTTAATGCCTTTGTATCATGCTATTGCTTCCCGTATGGCT
TTCATTTTCTCCTCCTTGTATAAATCCTGGTTGCTGTCTCTTTATGAGGAGTTGTGGCCCGTT
GTCAGGCAACGTGGCGTGGTGTGCACTGTGTTTGCTGACGCAACCCCCACTGGTTGGGGCATT
GCCACCACCTGTCAGCTCCTTTCCGGGACTTTCGCTTTCCCCCTCCCTATTGCCACGGCGGAA
CTCATCGCCGCCTGCCTTGCCCGCTGCTGGACAGGGGCTCGGCTGTTGGGCACTGACAATTCC
GTGGTGTTGTCGGGGAAGCTGACGTCCTTTCCATGGCTGCTCGCCTGTGTTGCCACCTGGATT
CTGCGCGGGACGTCCTTCTGCTACGTCCCTTCGGCCCTCAATCCAGCGGACCTTCCTTCCCGC
GGCCTGCTGCCGGCTCTGCGGCCTCTTCCGCGTCTTCGCCTTCGCCCTCAGACGAGTCGGATC
TCCCTTTGGGCCGCCTCCCCGCCTG
(SEQ ID NO: 22)
WPRE3
AATCAACCTCTGGATTACAAAATTTGTGAAAGATTGACTGGTATTCTTAACTATGTTGCTCCT
TTTACGCTATGTGGATACGCTGCTTTAATGCCTTTGTATCATGCTATTGCTTCCCGTATGGCT
TTCATTTTCTCCTCCTTGTATAAATCCTGGTTAGTTCTTGCCACGGCGGAACTCATCGCCGCC
TGCCTTGCCCGCTGCTGGACAGGGGCTCGGCTGTTGGGCACTGACAATTCCGTGGTGTT
(SEQ ID NO: 23)
WPREmut6delATG
GATAATCAACCTCTGGATTACAAAATTTGTGAAAGATTGACTGGTATTCTTAACTTTGTTGCT
CCTTTTACGCTTTGTGGATACGCTGCTTTATTGCCTTTGTATCTTGCTATTGCTTCCCGTTTG
GCTTTCATTTTCTCCTCCTTGTATAAATCCTGGTTGCTGTCTCTTTTTGAGGAGTTGTGGCCC
GTTGTCAGGCAACGTGGCGTGGTGTGCACTGTGTTTGCTGACGCAACCCCCACTGGTTGGGGC
ATTGCCACCACCTGTCAGCTCCTTTCCGGGACTTTCGCTTTCCCCCTCCCTATTGCCACGGCG
GAACTCATCGCCGCCTGCCTTGCCCGCTGCTGGACAGGGGCTCGGCTGTTGGGCACTGACAAT
TCCGTGGTGTTGTCGGGGAAATCATCGTCCTTTCCTTGGCTGCTCGCCTGTGTTGCCACCTGG
ATTCTGCGCGGGACGTCCTTCTGCTACGTCCCTTCGGCCCTCAATCCAGCGGACCTTCCTTCC
CGCGGCCTGCTGCCGGCTCTGCGGCCTCTTCCGCGTCTTCGCCTTCGCCCTCAGACGAGTCGG
ATCTCCCTTTGGGCCGCCTCCCCGCATCGGACTAG
(SEQ ID NO: 24)
CRE4
GAGTTAATGTGTAATGAGACGGGGAGAAAACAGGAGAGCCCAGAATGACCTGGATGCTGATCA
CTGAACATACCCTACCCCCAGTAAAACAAATTCAGAAAACAGCTTCCGCCCGTCCCCTCCCAA
TGGAGGGCTCTGGCAGGAAAAGAGGTGAATAAGAGGCTTCTACCAGGGTAAAGGTTGAAGGCA
CCTGGTCATTTGATCACCTTATCAGTTCTAGGCAGTGATTAGCCAATATTGAGTCAGCAGGGG
CAATAGCCCTGGCCCTTGTCTCACTCCTGTTGGGGGTGGGGGAGGGGGAGAGGTACATTCCCA
GGTTCAAAGCATTTGGGTGAAATCAGTTAAATAGATATCAGAAGCTTTTGTATCTTTCACCCT
TTTGCCCCCCAAGCATACTCGCTGAGTATGTGGAACATTCCTGAGGGT
(SEQ ID NO: 25)
CRE6
AGGGGGGCCCCTGGGGCTGACAGGGACTGGAAGCTCTGAGCTGGCCAGAGGGATGTTGCAATC
CTGCCAGGGTCTTGTCTATGCTGTCCTTTTCACAACCATCCCCCTACTGCCAGGCTGACACGT
GGTTGCGGGGGCACAAGGCCAGCCAACCTAGAGTCTGAGGCTAGGCGGAGGACACCCTCCCCA
CCAGCTGCCAGGGTCACTGGCGGTCAAAGGCAGCTGGTGGGGAAGGCATTGGACTCCAGCCTT
GGGGGACGGATGTAG
(SEQ ID NO: 26)
hTTR
AAATGACCTATTAAGAATATTTCATAGAACGAATGTTCCGATGCTCTAATCTCTCTAGACAAG
GTTCATATTTGTATGGGTTACTTATTCTCTCTTTGTTGACTAAGTCAATAATCAGAATCAGCA
GGTTTGCAGTCAGATTGGCAGGGATAAGCAGCCTAGCTCAGGAGAAGTGAGTATAAAAGCCCC
AGGCTGGGAGCAGCCATCACAGAAGTCCACTCATTCTTGGCAGG
(SEQ ID NO: 27)
hTTR, Wild type mTTR and modified mTTR (Costa et al. 1991)

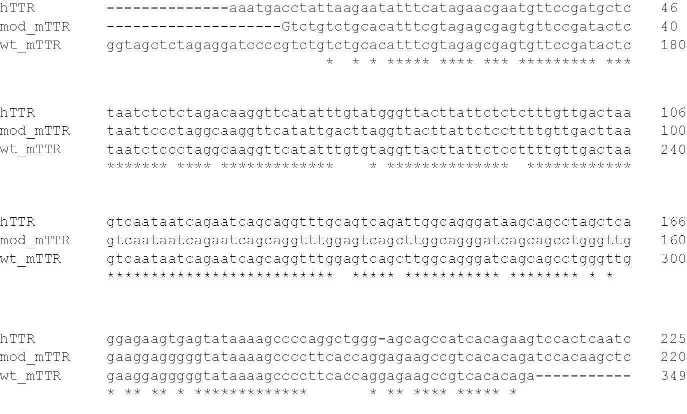

(mod_mTTR: SEQ ID NO: 28; wt_mTTR: SEQ ID NO: 29)

Sequence identity of codon-optimized SERPING1 sequences (HA03, HA04, HA05, and HA06) with the wild type SERPING1 sequence (HA01), and sequence identity of one codon-optimized SERPING1 sequence with the other codon-optimized sequence are given below in Table 3.

TABLE 3
Sequence identity among wild type and
codon-optimized SERPING1 sequences
Codon-optimization of SEREPING1:
Sequence Identity

| | HA04 | HA03 | HA06 | HA05 |
|---|---|---|---|---|
| HA01 | 97 | 83 | 90 | 93 |
| HA04 | | 84 | 92 | 96 |
| HA03 | | | 91 | 87 |
| HA06 | | | | 95 |

Use of rAAV Vectors Encoding C1-INH for Treatment of Disease

Described herein are methods of treating a disease associated with C1-INH deficiency. Accordingly, in some embodiments, the rAAV vectors described herein are suitable for treating a subject that has a C1-INH deficiency, such as patients suffering from HAE. The method of treating includes administering to the subject in need thereof a recombinant adeno-associated virus (rAAV) vector as described herein.

The rAAV vector described herein can be used to treat any disease associated with C1-INH deficiency or disorder.

In some embodiments, the rAAV vector remains episomal following administration to a subject in need thereof. In some embodiments, the rAAV vector does not remain episomal following administration to a subject in need thereof. For example, in some embodiments, the rAAV vector integrates into the genome of the subject. Such integration can be achieved, for example, by using various gene-editing technologies, such as, zinc finger nucleases (ZFNs), Transcription activator-like effector nucleases (TALENS), ARCUS genome editing, and/or CRISPR-Cas systems.

In some embodiments, a pharmaceutical composition comprising a rAAV vector described herein is used to treat subjects in need thereof. The pharmaceutical composition containing a rAAV vector or particle of the invention contains a pharmaceutically acceptable excipient, diluent or carrier. Examples of suitable pharmaceutical carriers are well known in the art and include phosphate buffered saline solutions, water, emulsions, such as oil/water emulsions, various types of wetting agents, sterile solutions and the like. The pharmaceutical composition can be in a lyophilized form. Such carriers can be formulated by conventional methods and are administered to the subject at a therapeutically effective amount.

The rAAV vector is administered to a subject in need thereof via a suitable route. In some embodiments, the rAAV vector is administered by intravenous, intraperitoneal, subcutaneous, or intradermal routes. In one embodiment, the rAAV vector is administered intravenously. In embodiments, the intradermal administration comprises administration by use of a “gene gun” or biolistic particle delivery system. In some embodiments, the rAAV vector is administered via a non-viral lipid nanoparticle. For example, a composition comprising the rAAV vector may comprise one or more diluents, buffers, liposomes, a lipid, a lipid complex. In some embodiments, the rAAV vector is comprised within a microsphere or a nanoparticle, such as a lipid nanoparticle or a inorganic nanoparticle.

In some embodiments, functional C1-INH is detectable in plasma of the subject at about 1 to 6 weeks post administration of the rAAV vector. In some embodiments, functional C1-INH is detectable in plasma of the subject at about one week. In some embodiments, functional C1-INH is detectable in plasma of the subject at about 2 weeks. In some embodiments, functional C1-INH is detectable in plasma of the subject at about 3 weeks. In some embodiments, functional C1-INH is detectable in plasma of the subject at about 4 weeks. In some embodiments, functional C1-INH is detectable in plasma of the subject at about 5 weeks. In some embodiments, functional C1-INH is detectable in plasma of the subject at about 6 weeks. In some embodiments, functional C1-INH is detectable in hepatocytes of the subject at about 1 to 6 weeks post administration of the rAAV vector. In some embodiments, functional C1-INH is detectable in hepatocytes of the subject greater than 7 weeks post administration of the rAAV vector.

In some embodiments, functional C1-INH is detectable in plasma of the subject at least 1 month, 2 months, 3 months, 4 months, 5 months, 6 months, 12 months, 2 years, 3 years, 4 years, 5 years, 6 years, 7 years, 8 years, 9 years, or 10 years after administration of the rAAV vector. Accordingly, in some embodiments, functional C1-INH is detectable in plasma of the subject at least 3 months after administration of the rAAV vector. In some embodiments, functional C1-INH is detectable in plasma of the subject at least 6 months after administration of the rAAV vector. In some embodiments, functional C1-INH is detectable in plasma of the subject at least 12 months after administration of the rAAV vector. In some embodiments, functional C1-INH is detectable in plasma of the subject at least 2 years after administration of the rAAV vector. In some embodiments, functional C1-INH is detectable in plasma of the subject at least 3 years after administration of the rAAV vector. In some embodiments, functional C1-INH is detectable in plasma of the subject at least 4 years after administration of the rAAV vector. In some embodiments, functional C1-INH is detectable in plasma of the subject at least 5 years after administration of the rAAV vector. In some embodiments, functional C1-INH is detectable in plasma of the subject at least 6 years after administration of the rAAV vector. In some embodiments, functional C1-INH is detectable in plasma of the subject at least 7 years after administration of the rAAV vector. In some embodiments, functional C1-INH is detectable in plasma of the subject at least 8 years after administration of the rAAV vector. In some embodiments, functional C1-INH is detectable in plasma of the subject at least 9 years after administration of the rAAV vector. In some embodiments, functional C1-INH is detectable in plasma of the subject at least 10 years after administration of the rAAV vector. In some embodiments, functional C1-INH is detectable in plasma of the subject for the remainder of the subject's life following administration of the rAAV vector.

In some embodiments, the administered rAAV comprising SERPING1 results in the production of active C1-INH to the same extent as found following administration of purified C1-INH protein delivered intravenously. In some embodiments, the administered rAAV comprising SERPING1 results in production of a greater amount of active C1-INH as compared to administration of purified C1-INH protein delivered intravenously.

In some embodiments, the administered rAAV comprising SERPING1 results in the increase of C1-INH in the subject. In some embodiments, the increase of C1-INH is detected in plasma of the subject. In some embodiments, the increase of C1-INH is detected in liver tissue of the subject. In some embodiments, the increase of C1-INH can be detected in one or more tissues/organs including gall bladder, spleen, ovary, urinary bladder, fat, placenta, lung, prostate, heart, lymph node, and endometrium. In some embodiments, the administered rAAV comprising SERPING1 increases C1-INH in the subject by about 95%, 90%, 85%, 80%, 75%, 70%, 65%, 60%, 55%, 50%, 45%, 40%, 35%, 30%, 25%, 20%, 15%, or about 10% in comparison to the subject's baseline C1-INH level prior to administering the rAAV comprising SERPING1. Accordingly, in some embodiments, the administered rAAV comprising SERPING1 increases C1-INH in the subject by about 95%. In some embodiments, the administered rAAV comprising SERPING1 increases C1-INH in the subject by about 90%. In some embodiments, the administered rAAV comprising SERPING1 increases C1-INH in the subject by about 85%. In some embodiments, the administered rAAV comprising SERPING1 increases C1-INH in the subject by about 80%. In some embodiments, the administered rAAV comprising SERPING1 increases C1-INH in the subject by about 75%. In some embodiments, the administered rAAV comprising SERPING1 increases C1-INH in the subject by about 70%. In some embodiments, the administered rAAV comprising SERPING1 increases C1-INH in the subject by about 65%. In some embodiments, the administered rAAV comprising SERPING1 increases C1-INH in the subject by about 60%. In some embodiments, the administered rAAV comprising SERPING1 increases C1-INH in the subject by about 55%. In some embodiments, the administered rAAV comprising SERPING1 increases C1-INH in the subject by about 50%. In some embodiments, the administered rAAV comprising SERPING1 increases C1-INH in the subject by about 45%. In some embodiments, the administered rAAV comprising SERPING1 increases C1-INH in the subject by about 40%. In some embodiments, the administered rAAV comprising SERPING1 increases C1-INH in the subject by about 35%. In some embodiments, the administered rAAV comprising SERPING1 increases C1-INH in the subject by about 30%. In some embodiments, the administered rAAV comprising SERPING1 increases C1-INH in the subject by about 25%. In some embodiments, the administered rAAV comprising SERPING1 increases C1-INH in the subject by about 20%. In some embodiments, the administered rAAV comprising SERPING1 increases C1-INH in the subject by about 15%. In some embodiments, the administered rAAV comprising SERPING1 increases C1-INH in the subject by about 10%.

In some embodiments, following administration of the AAV vector to the subject the levels of functional C1-INH detectable in the circulation are between about 2 and 20 times greater than the amount of functional C1-INH detectable in the subject before administration of the rAAV comprising SERPING1.

In some embodiments, following administration of the AAV vector to the subject the levels of detectable active C1-INH meets or exceeds human therapeutic level. In some embodiments, the levels of active C1-INH post administration of the rAAV vector is about between 2 and 35 times the human therapeutic level. In some embodiments, the levels of active C1-INH post administration is about 2 times the human therapeutic level. In some embodiments, the levels of active C1-INH post administration is about 3 times the human therapeutic level. In some embodiments, the levels of active C1-INH post administration is about 4 times the human therapeutic level. In some embodiments, the levels of active C1-INH post administration is about 5 times the human therapeutic level. In some embodiments, the levels of active C1-INH post administration is about 6 times the human therapeutic level. In some embodiments, the levels of active C1-INH post administration is about 6 times the human therapeutic level. In some embodiments, the levels of active C1-INH post administration is about 7 times the human therapeutic level. In some embodiments, the levels of active C1-INH post administration is about 8 times the human therapeutic level. In some embodiments, the levels of active C1-INH post administration is about 9 times the human therapeutic level. In some embodiments, the levels of active C1-INH post administration is about 10 times the human therapeutic level. In some embodiments, the levels of active C1-INH post administration is about 15 times the human therapeutic level. In some embodiments, the levels of active C1-INH post administration is about 20 times the human therapeutic level. In some embodiments, the levels of active C1-INH post administration is about 25 times the human therapeutic level. In some embodiments, the levels of active C1-INH post administration is about 30 times the human therapeutic level. In some embodiments, the levels of active C1-INH post administration is about 35 times the human therapeutic level.

In some embodiments, the rAAV. SERPING1 vector is delivered as a single dose per subject. In some embodiments, the subject is delivered the minimal effective dose (MED). As used herein, MED refers to the rAAV SERPING1 vector dose required to achieve C1-INH activity resulting in increased C1-INH levels in a subject.

The vector titer is determined on the basis of the DNA content of the vector preparation. In some embodiments, quantitative PCR or optimized quantitative PCR is used to determine the DNA content of the rAAV SERPING1 vector preparations. In one embodiment, the dosage is about 1×10¹¹ vector genomes (vg)/kg body weight to about 2×10¹³ vg/kg, inclusive of endpoints.

In some embodiments, the dosage is at least 5×10⁹ vg/kg or above.

In some embodiments, the rAAV SERPING1 vector compositions can be formulated in dosage units to contain an amount of replication-defective virus that is in the range of about 1.0×10⁹ vg to about 1.0×10¹⁵ vg. As used herein, the term “dosage” can refer to the total dosage delivered to the subject in the course of treatment, or the amount delivered in a single (of multiple) administration.

In some embodiments, the dosage is sufficient to increase plasma C1-INH levels in the patient by 25% or more. In some embodiments, rAAV SERPING1 is administered in combination with one or more therapies for the treatment of HAE.

### EXAMPLES

Other features, objects, and advantages of the present invention are apparent in the examples that follow. It should be understood, however, that the examples, while indicating embodiments of the present invention, are given by way of illustration only, not limitation. Various changes and modifications within the scope of the invention will become apparent to those skilled in the art from the examples.

### Example 1. Vector Design

Exemplary methods and designs of generating rAAV expression constructs (rAAV vectors) comprising coding sequences of human C1-esterase inhibitor (C1-INH) and variations of the same are provided in this Example. In this study, hSERPING1 was used as a coding sequence for human C1-INH (hC1-INH), and recombinant AAV vector (rAAV8) was used as a vector. The basic design of a rAAV vector comprises an expression cassette flanked by inverted terminal repeats (ITRs): a 5′-ITR and a 3′-ITR These ITRs mediate the replication and packaging of the vector genome by the AAV replication protein Rep and associated factors in vector producer cells. Typically, an expression cassette contains a promoter, a coding sequence, a polyA tail and/or a tag, as shown in FIG. 1A. An expression construct hSERPING1 encoding human hC1-INH was designed and prepared using standard molecular biology techniques. The coding sequence for the hSERPING1 was inserted downstream of a promoter, hTTR (human transthyrethin promoter). Additionally, a liver-specific cis-acting regulatory module (CRM) was inserted upstream of the promoter, and an intron sequence was inserted downstream of the promoter. This regulatory and promoter combination was tested for high transduction level, as shown in the examples that follow. The expression construct was then ligated to the AAV vector and tested by sequencing. Vectors were packaged in viral particles and stored.

In another embodiment, a WPRE sequence was inserted downstream of the coding region. This element creates a tertiary structure that increases the mRNA stability. A schematic representation of the expression constructs described herein is shown in FIG. 1B. Any number of variations of the above scheme can be performed.

Condon-Optimized Constructs

Additionally, the coding sequences for the SERPING1 were codon-optimized based on multiple parameters, such as codon adaptation index (CAI), CpG site count, GC content, and repetitious base sequences. High CAI was preferred to utilize more frequently used codons and to potentially increase transgene product expression level from the vector. CpG island sequences, which can elicit immune response, were reduced. Repetitious bases were also removed. Any number of variations of the above scheme can be performed. For example, more than one promoter may be used. Additionally, different combinations of regulatory region, promotor, intron, and exon can be contemplated.

TABLE 4
Examples of different expression constructs with different codon-optimized
SERPING1 coding sequences and associated elements.

| Expression | Coding | | | Promoter | Other |
|---|---|---|---|---|---|
| Constructs | Sequences | Enhancer | Intron | elements | elements |
| M constructs | wild type | | SERPING1- | hTTR | |
| (Two sequences) | SERPING1 | | specific | | |
| | coding sequence | | intron or | | |
| | (HA01) | | MVM | | |
| | | | intron | | |
| J constructs | codon- | | MVM | hTTR | |
| (Four different | optimized | | intron | | |
| sequences) | SERPING1 | | | | |
| | (HA03, HA04, | | | | |
| | HA05, and | | | | |
| | HA06) | | | | |
| S constructs | codon- | WPRE, WPRE3, | MVM | hTTR | |
| (Six different | optimized | or, | intron, or | | |
| sequences) | SERPING1 | WPREmut6delATG | Exon1- | | |
| | (HA06) | | Intron1- | | |
| | | | partially | | |
| | | | Exon2 | | |
| N constructs | codon- | | MVM | hTTR, | |
| (Five different | optimized | | intron | hTTR | |
| sequences) | SERPING1 | | | mod., | |
| | (HA06) | | | mTTR | |
| | | | | pro, | |
| | | | | mTTR | |
| | | | | enhancer, | |
| | | | | CAG | |
| | | | | promoter, | |
| | | | | or | |
| | | | | CMV/CBA | |
| | | | | promoter | |
| U constructs | optimized | WPREmut6delATG | MVM | hTTR | |
| (Ten different | SERPING1 | | intron | | |
| sequences) | sequences | | | | |
| | (HA11, HA12, | | | | |
| | HA13, . . . HA20) | | | | |
| P constructs | codon- | WPREmut6delATG | MVM | hTTR | CRE4, |
| (Six different | optimized | | intron | | or/and |
| sequences) | SERPING1 | | | | CRE6, |
| | (HA06) | | | | |

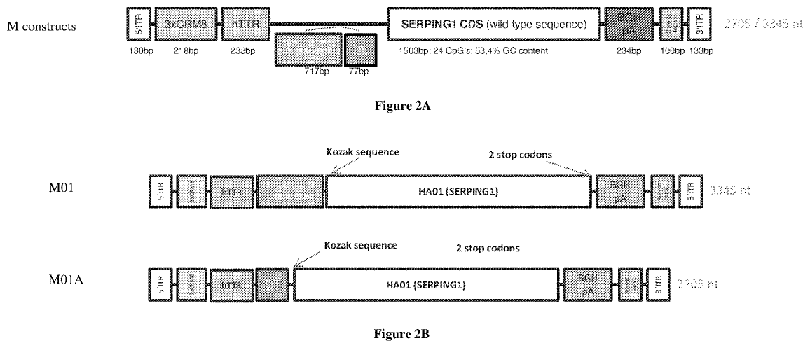

Different expression constructs designed for treating human angioedema (HAE constructs) are listed in Table 4, and have been depicted in FIGS. 2A-7B. M constructs, in general, are shown in FIG. 2A, and two M constructs—M01 and M01A—are shown in FIG. 2B. Both M01 and M01A constructs include HA01 sequence which is a human SERPING1 wild type sequence (a control construct). HA01 includes 24 CpG's and 53.4% GC content. M01 includes an intron-SERPING1 exon 1-intron 1-partially exon 2 (717 bp), while M01A includes MVM (77 bp) intron.

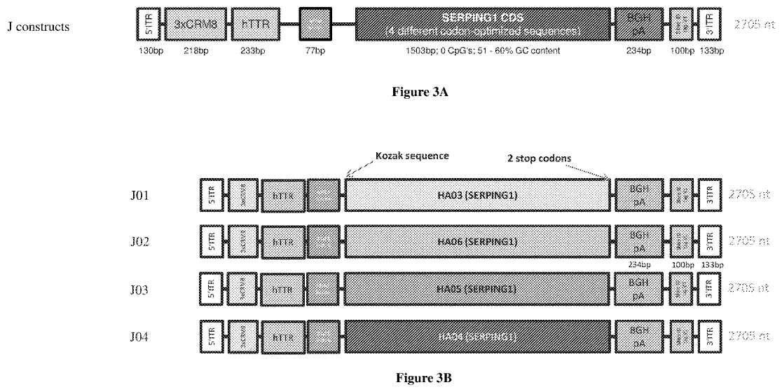

J constructs, in general, are shown in FIG. 3A, and four J constructs—J01, J02, J03, and J04, are shown in FIG. 3B. J constructs are codon-optimized SERPING1 sequences. J01 includes HA03 SERPING1 sequence; J02 includes HA06 SERPING1 sequence; J03 includes HA05 SERPING1 sequence; and J04 includes HA04 SERPING1 sequence.

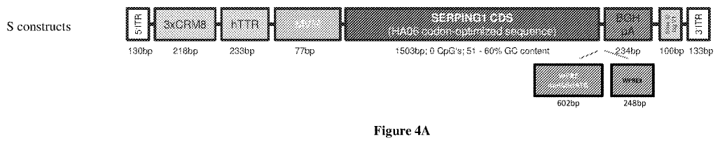

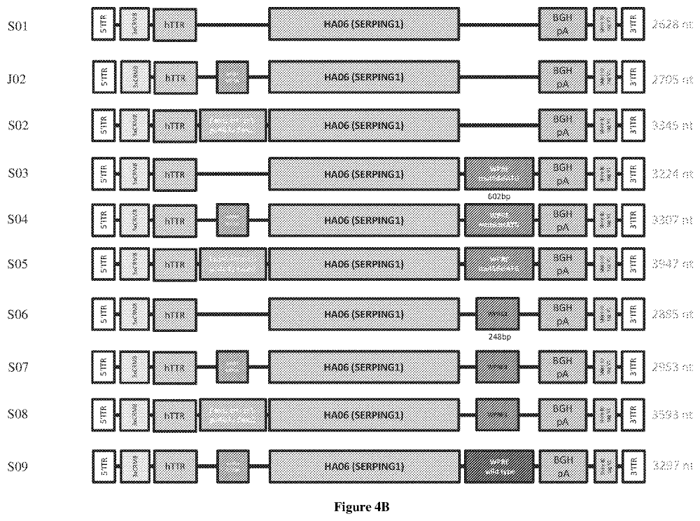

S constructs, in general, are shown in FIG. 4A, and nine S constructs—S01, S02, S03, S04, S05, S06, S07, S08, and S09 are shown in FIG. 4B. S constructs include HA06 SERPING1 sequence, intron (MVM intron or Exon1-Intron1-partially Exon2), and WPRE (either WPRE3 or WPREmut6delATG).

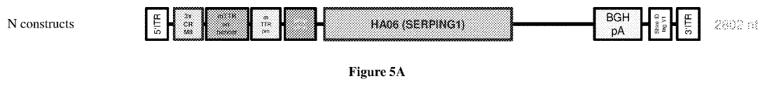

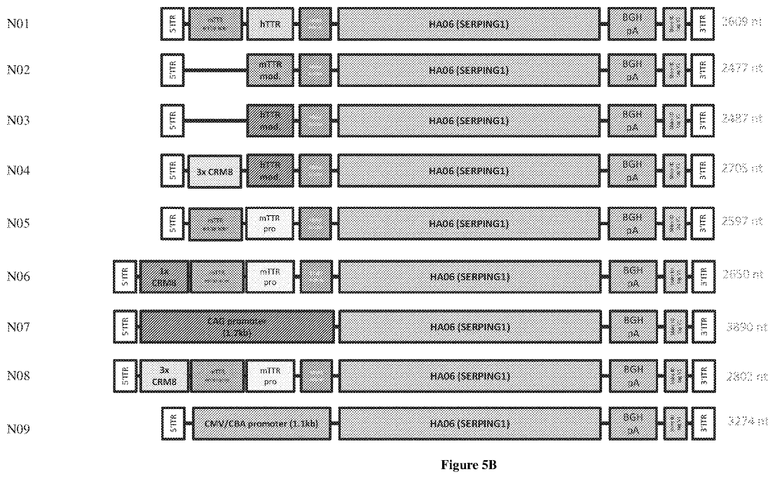

N constructs, in general, are shown in FIG. 5A, and nine N constructs—Naptune01, Naptune02, Naptune03, Naptune04, Naptune05, Naptune06, Naptune07, Naptun08, and Naptun09 are shown in FIG. 5B. N constructs includes HA06 SERPING1 sequence and novel promoter elements such as hTTR, hTTR mod., mTTR pro, mTTR enhancer, CAG promoter, or CMV/CBA promoter.

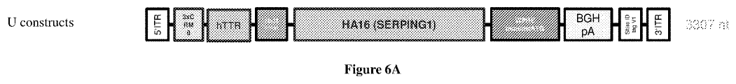

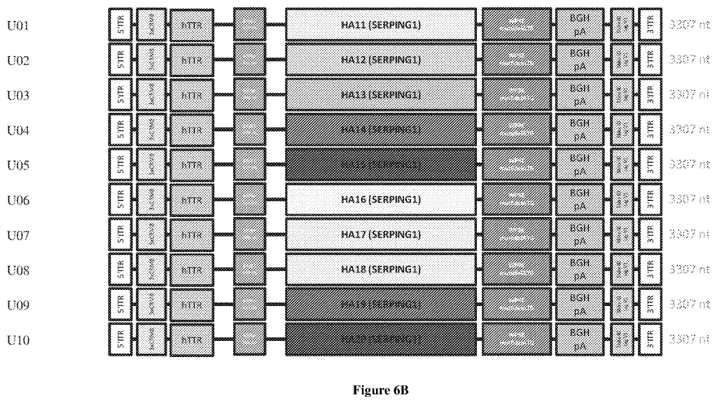

U constructs, in general, are shown in FIG. 6A, and ten U constructs—U01, U02, U03, U04, U05, U06, U07, U08, U09, and U10 are shown in FIG. 6B. U constructs include codon-optimized sequences and WPREmut6delATG.

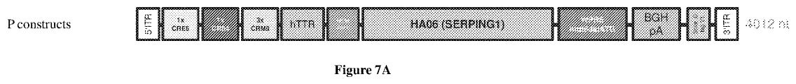

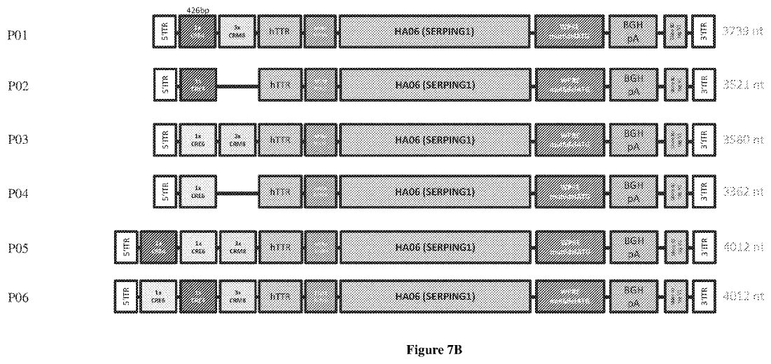

P constructs, in general, are shown in FIG. 7A, and six P constructs—P01, P02, P03, P04, P05, and P06 are shown in FIG. 7B. P constructs include HA06 SERPING1 sequence WPREmut6delATG, and modified backbone elements CRE4 and/or CRE6.

### Example 2. AAV8.SERPING1 Vector Mediated Expression of Glycosylated and Functional hC1-INH In Vitro

This example illustrates the potency of the AAV8.SERPING1 vector in hC1-INH expression in vitro.

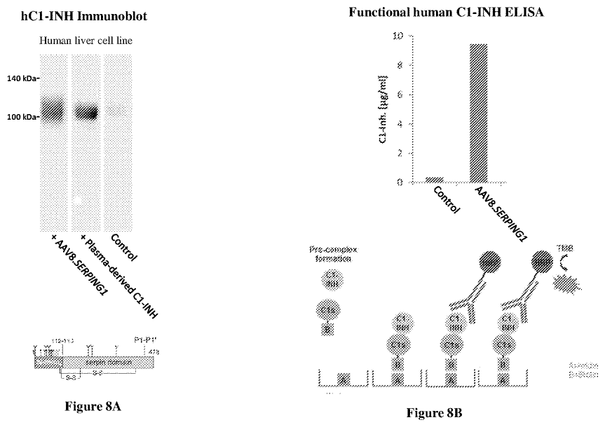

HepG2 cells (liver cells) were transfected with rAAV vectors (AAV8.SERPING1) expressing hC1-INH or with a control vector (as a negative control), and supernatants were collected after 72 h. The rAAV vector construct is depicted in FIG. 1A. A plasma-derived hC1-INH sample was used as a positive control. The hC1-INH expression in cell supernatants was evaluated on an immunoblot using standard Western Blot analysis. As shown in FIG. 8A, hC1-INH was detected in the supernatant obtained from rAAV vectors treated cells. The results from this example show the expression of hC1-INH from the rAAV vector.

The level of hC1-INH expression in HepG2 cells was determined by measuring the amount of hC1-INH present in the supernatants using ELISA. As shown in FIG. 8B, rAAV transfected cells expressed significantly high amount of hC1-INH compared to the control cells. The results from this example show that rAAV transfected cells express functional hC1-INH.

### Example 3. Dose-Dependent AAV8.hSERPING1 Vector Mediated Expression of hC1-INH In Vivo

This example illustrates the potency of rAAV (i.e AAV8.hSERPING1) vector in dose-dependent expression of hC1-INH in vivo.

To demonstrate in vivo expression, an AAV8.hSERPING1vector encoding hC1-INH, depicted as M01 in FIG. 2B, was injected into mice (C57/bl/6) intravenously. Three different doses—1×10¹¹ vg/kg, 4×10¹¹ vg/kg, and 4×10¹² vg/kg—of vector were evaluated. Each mice received a single dose. Plasma samples were collected 14 days post injection.

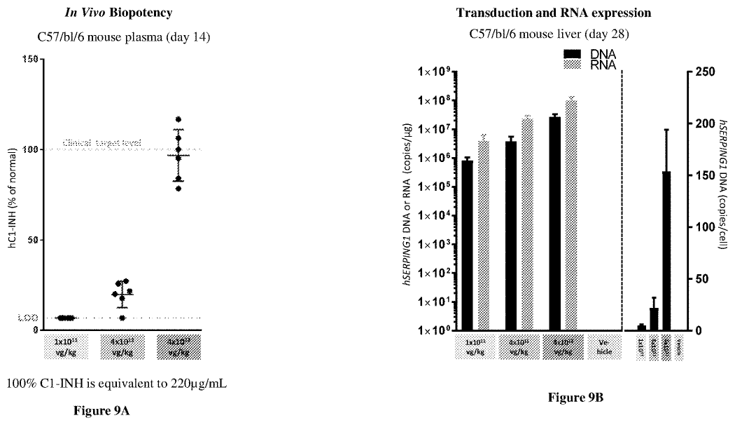

The efficacy of the rAAV was determined by monitoring levels of hC1-INH in plasma. The level of hC1-INH in plasma was expressed in terms of the percentage of hC1-INH level present in a normal mice. Results are depicted in FIG. 9A. Mice administered with the rAAV vector showed expression of hC1-INH in a dose-dependent manner. The group of mice that received rAAV vectors at the dose of 4×10¹² vg/kg expressed a level of hC1-INH equivalent to that of a clinical target level (i.e. 220 μg/mL).

Furthermore, the transduction and transcription efficiencies of the rAAV were also determined by injecting the vectors into mice intravenously. Mice (C57/bl/6) received the rAAV at one of the three doses—1×10¹¹ vg/kg, 4×10¹¹ vg/kg, and 4×10¹² vg/kg, and a fourth group of mice received the vehicle only. The rAAV vector construct is depicted as M01 in FIG. 2B. Twenty eight days post injection, the animals were sacrificed, and their livers were harvested. The transduction and transcription efficiencies of the rAAV were compared with that of the group that received the vehicle only. The results are presented in FIG. 9B, and show that there was significant transduction and transcription of hSERPING1 at all three doses. In fact, a dose-dependent enhanced transduction was observed when hSERPING1 DNA was expressed in terms of hSERPING1 DNA copy/cell. As expected, there was no transduction and transcription of hSERPING1 in mice that received the vehicle only.

The results from this example show that rAAV.AAV8.hSERPING1 vectors express C1-INH in vivo in a dose-dependent manner. This result also reveals the presence of physiological human C1-INH levels in wild-type mice.

### Effect of Codon-Optimization and Screening of Codon-Optimized Constructs

### Example 4. In Vivo Efficacy of rAAV8 Vectors with Codon-Optimized hSERPING1 Sequences

This example illustrates in vivo efficacies of some codon-optimized rAAV8.C1-INH constructs in expressing C1-INH in plasma.

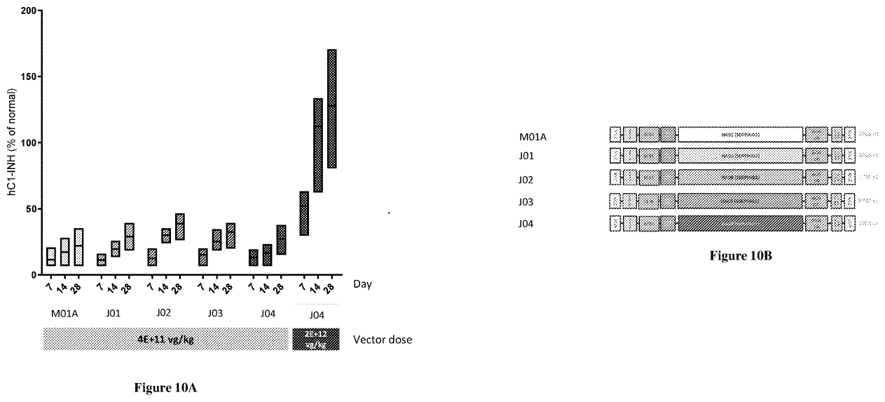

In order to test the effect of codon-optimization of hSERPING1 sequences on the in vivo efficacy of rAAV8 vectors, four codon-optimized constructs were prepared. The codon-optimized constructs were—J01, J02, J03, and J04. Mice (C57bl/6) were injected with rAAV vectors comprising either M01A (a wild-type C1-INH) or codon-optimized C1-INH sequences. All five constructs are depicted in FIG. 10B. Mice received either 4×10¹¹ vg/kg or 2×10¹² vg/kg of vectors, and plasma samples were collected prior to administration of rAAV and at day 7, day 14, and day 28 post injection. The dose response was only evaluated using J04. In this study only male mice were used. The level of hC1-INH in plasma was expressed in terms of the percentage of hC1-INH level present in a normal mouse. Results are depicted in FIG. 10A and in Table 5.

Mice administered with control vector (wild-type) as well as codon-optimized constructs expressed hC1-INH. Different constructs showed a moderate effect of codon optimization on their ability to express hC1-INH.

TABLE 5
Expression of hC1-INH by rAAV comprising codon-optimized
SERPING1 sequences compared to that of vectors
comprising wild-type SERPING1

| | | | | Fold expression |
|---|---|---|---|---|
| | | | | compared to |
| | | | | wild-type on day |
| Group | Condition | Treatment | Vector Dose | 14 |
| A | Control | M01A | 4 × 10¹¹ vg/kg | 1 |
| | vector | | | |
| B | Test | J01 | 4 × 10¹¹ vg/kg | 1.1 |
| C | Test | J02 | 4 × 10¹¹ vg/kg | 1.5 |
| D | Test | J03 | 4 × 10¹¹ vg/kg | 1.5 |
| E | Test | J04 | 4 × 10¹¹ vg/kg | 1.2 |

The codon-optimized construct, J04, expressed hC1-INH in a dose-dependent manner throughout the study (i.e at day 7, at day 14, and at day 28), as demonstrated in FIG. 10A.

The results from this example show that codon optimization moderately improves the efficacy of a construct over the wild-type (control vector). The codon-optimized constructs also express hC1-INH in a dose-dependent manner. Based on this study, J02 was selected for subsequent in-life studies.

Effect of WPRE

### Example 5. In Vivo Efficacy of a rAAV8 Vector Comprising Codon-Optimized hSERPING1 Sequences and WPRE

This example illustrates the in vivo efficacy of a vector comprising a codon-optimized AAV8.C1-INH-co2 sequence in combination with three different WPREs in expressing C1-INH in plasma.

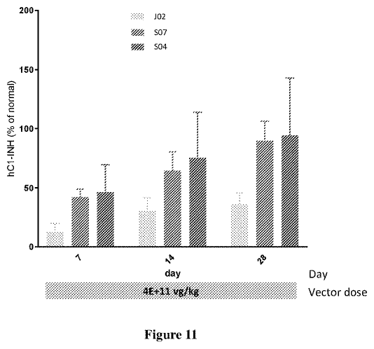

Mice were injected with rAAV vectors comprising different codon-optimized constructs: (1) J02 (no WPRE element); (2) S07 (containing WPRE3); and S04 (containing WPREmut6delATG). The rAAV vector comprising WPRE is depicted in FIG. 1B, and the different codon-optimized constructs and associated elements are listed in Table 4. Mice received 4×10¹¹ vg/kg of vectors, and plasma samples were collected prior to administration of rAAV and at day 7, day 14, and day 28 post injection. The level of hC1-INH in plasma was expressed in terms of the percentage of hC1-INH level present in a normal mouse. Results are depicted in FIG. 11.

Mice administered with the construct comprising WPRE expressed about 2.5 fold more hC1-INH compared to a codon-optimized constructs without WPRE.

The results from this example show that the incorporation of WPRE improves the efficacy of the codon-optimized constructs.

### Effect of WPRE and Intron

### Example 6. In Vivo Efficacy of a rAAV8 Vector Comprising a HA06 (hSERPING1) Sequence, a WPRE, and an Intron

This example illustrates the in vivo efficacy of a rAAV8 vector comprising a HA06 (hSERPING1) sequence, a WPRE, and an intron for the expression of hC1-INH in plasma.

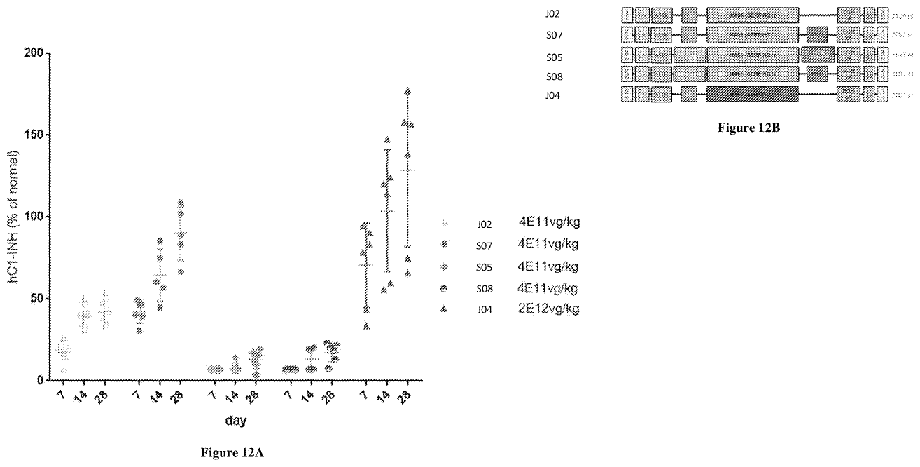

In order to test the effect of WPRE and intron on the in vivo efficacy of a rAAV8 vector comprising a HA06 sequence, combinations of different WPREs and introns were used. Introns used in this study were either an MVM intron or an exon 1-intron 1-partially exon 2, while the WPREs used were either a WPRE3 or a WPREmut6delATG. Recombinant AAV vectors comprising different combination of WPREs, introns, and codon-optimized hSERPING1 sequences used in this study are depicted in FIG. 12B. Mice (C57bl/6; male) received either 4×10¹¹ vg/kg or 2×10¹² vg/kg of vectors, and plasma samples were collected prior to administration of rAAV and at day 7, day 14, and day 28 post injection. The level of hC1-INH in plasma was expressed in terms of the percentage of hC1-INH level present in a normal mouse. Results are depicted in FIG. 12A.

Mice administered with the construct comprising MVM intron, HA06 (SERPING1) sequence and WPRE3 (for example, S07) expressed more hC1-INH compared to the remaining animals that received other constructs at the same dose. Mice that received J04 at 2×10¹² vg/kg showed the highest expression of hC1-INH.

The results from this example show that the incorporation of MVM intron and WPRE3 improves the efficacy of the HA06 (SERPING1) sequences.

### Effect of WPRE and Alternative Promoter Elements at Low and High Vector Doses

### Example 7. In Vivo Efficacy of a rAAV8 Vector Comprising HA06 (hSERPING1) Sequence, a WPRE and an Alternative Promoter

This example illustrates the in vivo efficacy of constructs comprising a HA06 (hSERPING1) sequence, a WPRE and a novel promoter in plasma.

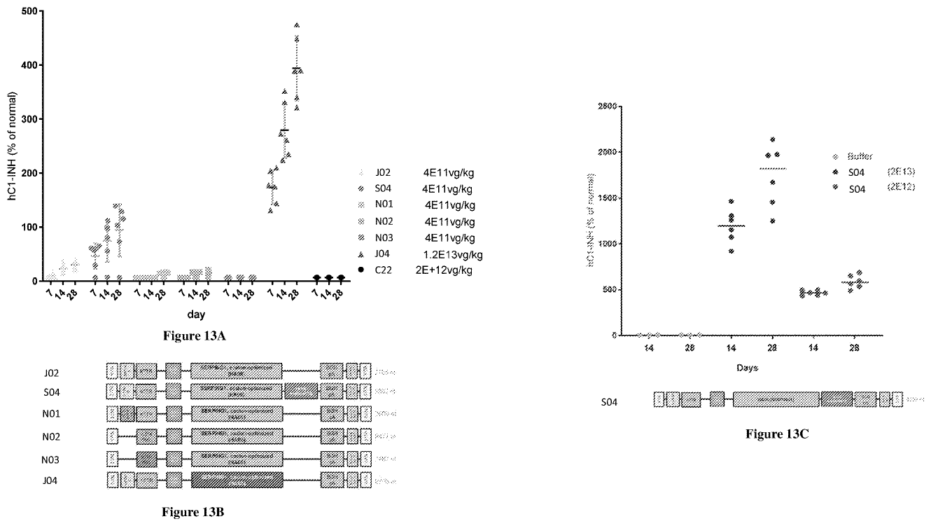

In order to test the effect of WPRE and other novel promoters on the in vivo efficacy of rAAV8 vectors, different constructs with different novel promoter elements were used. Seven different constructs—J02, J04, S04, N01, N02, N03, and C22—were tested. C22 included in this test is a control vector. All seven constructs are depicted in FIG. 13B. Mice (C57bl/6; male) received one of the three doses—4×10¹¹ vg/kg, 1.2×10¹³ vg/kg, and 2×10¹² vg/kg of vectors, and plasma samples were collected prior to administration of rAAV and at day 7, day 14, and day 28 post injection. The level of hC1-INH in plasma was expressed in terms of the percentage of hC1-INH level present in a normal mouse. Results are depicted in FIG. 13A.

Mice administered with the construct comprising HA06 (SERPING1) sequence and WPREmut6delATG (for example, S04) expressed more hC1-INH compared to the remaining animals that received other constructs at the same dose. Mice that received J04 at 1.2×10¹³ vg/kg showed the highest expression of hC1-INH. The results from this example show that the incorporation of WPREmut6delATG improves the efficacy of the HA06 (SERPING1) sequence.

In another example, mice (C57bl/6; male) received S04 at two doses—2×10¹² vg/kg and 2×10¹³ vg/kg, and plasma was collected at Day 14 and Day 28. Another group of mice received buffer to serve as a control. Results are depicted in FIG. 13C.

The results from this example show that S04 expresses hC1-INH in a dose dependent manner at both time-points.

### Effect of Codon-Optimization

### Example & In Vivo Efficacy of a rAAV8 Vector Comprising Constructs that Include Codon-Optimized hSERPING1 Sequencer

This example illustrates the in vivo efficacy of rAAV8 vectors comprising constructs that include codon-optimized hSERPING1 sequences in plasma.

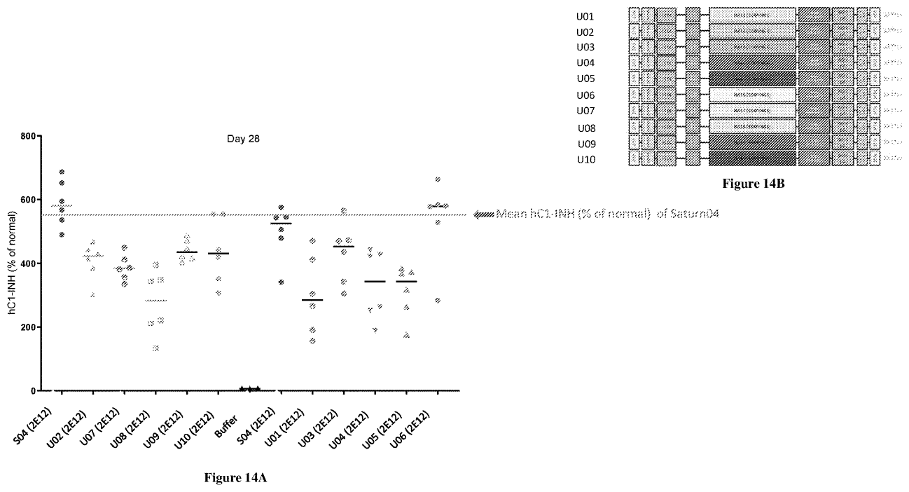

Ten different codon-optimized constructs—U01, U02, U03, U04, U05, U06, U07, U08, U09, and U10—were prepared. All ten constructs are depicted in FIG. 14B. The in vivo efficacy of all codon-optimized constructs were tested along with S04. The construct S04 is depicted in FIG. 4B. Mice (C57bl/6; male) received 2×10¹² vg/kg of vectors, and plasma samples were collected prior to administration of rAAV and at day 28 post injection. The level of hC1-INH in plasma was expressed in terms of the percentage of hC1-INH level present in a normal mouse. Results are depicted in FIG. 14A.

Mice administered with U06 expressed hC1-INH equivalent to that of group that received S04 at the same dose.

### Effect of WPRE and Alternative Promoter Elements at a Medium Dose in a Long Term Study

### Example 9. In Vivo Efficacy of a rAAV8 Vector Comprising Constructs that Include HA06 (hSERPING1) Sequence, a WPRE and an Alternative Promoter

This example illustrates the in vivo efficacy of rAAV8 vectors comprising constructs that include HA06 (hSERPING1) sequence, a WPRE and novel promoter elements in plasma.

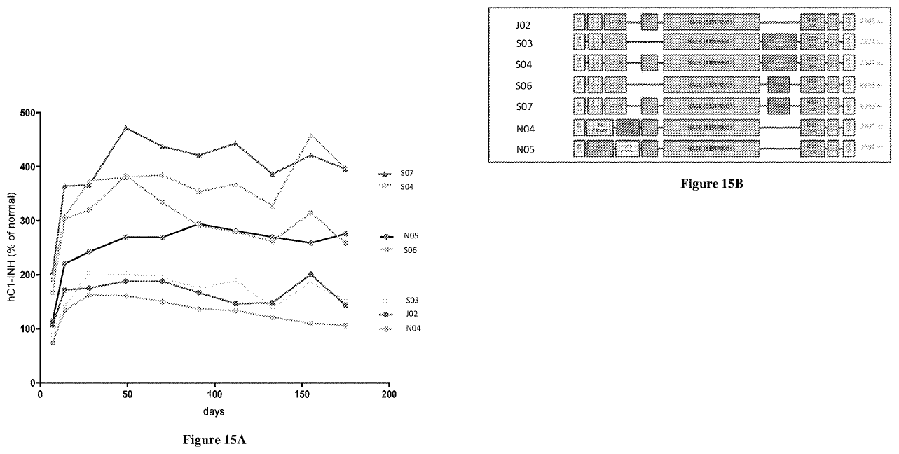

In order to test the effect of WPRE and novel promoters on the in vivo efficacy of rAAV8 vectors, different constructs with or without WPRE and novel promoter elements were used. Seven different constructs—J02, S03, S04, S06, S07, N04, and N05—were tested. All seven constructs are depicted in FIG. 15B. Mice (C57bl/6; male) received a medium dose of 2×10¹² vg/kg, and plasma samples were collected prior to administration of rAAV and at day 7, day 14, day 28, day 49, day 70, day 91, day 112, day 133, day 152, and day 175 post injection. The level of hC1-INH in plasma was expressed in terms of the percentage of hC1-INH level present in a normal mouse. Results are depicted in FIG. 15A.

Mice administered with the construct comprising MVM intron (for example, S07) expressed more hC1-INH compared to the group that received a construct without an MVM (for example, S06). In addition, the group that received construct comprising a shorter WPRE, a WPRE3, (for example, S06 and S07) expressed more hC1-INH compared to the group that received a construct comprising modified WPRE, WPREmut6delATG (for example, S03 and S04).

The results from this example show that the incorporation of MVM intron and a shorter form of WPRE, WPRE3, improve the efficacy of the HA06 (SERPING1) sequence. The construct, S07 that incorporates both a MVM intron and WPRE3 demonstrates the highest efficacy to express hC1-INH over 175 days.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A recombinant adeno-associated virus (rAAV) vector comprising an AAV8 capsid and a codon-optimized SERPING1 sequence encoding a C1 inhibitor (C1-INH) having at least about 70% identity to SEQ ID NO: 2.

2.-3. (canceled)

4. The rAAV vector of claim 1, wherein the vector further comprises a liver-specific promoter, wherein the liver-specific promoter is transthyretin promoter (TTR).

5.-6. (canceled)

7. The rAAV vector of claim 1, wherein the vector further comprises one or more of the following: a 5′ and a 3′ inverted terminal repeat, an intron upstream of the sequence, and a cis-acting regulatory module (CRM).

8. The rAAV vector of claim 1, wherein the vector further comprises a WPRE sequence.

9. The rAAV vector of claim 1, wherein the vector further comprises a modified WPRE sequence.

10. The rAAV vector of claim 1, wherein the vector further comprises a WPRE sequence, wherein the WPRE sequence contains a mut6delATG modification.

11. The rAAV vector of claim 1, wherein the vector comprises an intron, wherein the intron is a minute virus of mice (MVM) or SV40 intron.

12. The rAAV vector of claim 1, wherein the vector comprises a CRM, wherein the CRM is liver-specific CRM.

13. The rAAV vector of claim 1, wherein the vector further comprises a CRM, wherein the CRM is CRM8.

14. The rAAV vector of claim 1, wherein the vector further comprises at least three CRMs.

15. A recombinant adeno-associated virus (rAAV) comprising an AAV8 capsid and an rAAV vector, said vector comprising:
a. a 5′ inverted terminal repeat (ITR);
b. a cis-acting regulatory module (CRM);
c. a liver specific promoter;
d. a minute virus of mice (MVM);
e. a SERPING1 sequence encoding C1 inhibitor (C1-INH);
f. a woodchuck hepatitis virus posttranscriptional regulatory element (WPRE); and
g. a 3′ ITR.

16. The rAAV of claim 15, wherein the SERPING1 sequence is a wild type sequence or a codon-optimized sequence, having at least about 70% identity with SEQ ID NO: 2.

17.-18. (canceled)

19. A method of treating a subject having hereditary angioedema (HAE), comprising administering to the subject in need thereof a recombinant adeno-associated virus (rAAV) vector comprising an AAV8 capsid, and a promoter operably linked to a nucleic acid sequence that encodes C1 inhibitor (C1-INH), and wherein administering results in an increase in C1-INH enzymatic activity in the subject.

20. The method of claim 19, wherein the C1-INH is detected in the plasma of the subject.

21. The method of claim 19, wherein the C1-INH is detected in the liver of the subject.

22. The method of claim 19, wherein C1-INH is maintained for at least 30, 60, 90, 120, 150, 180 days or more after a single administration.

23. (canceled)

24. The method of claim 19, wherein the subject has C4 level restored to a pre-attack level.

25. The method of claim 19, wherein the AAV is administered intravenously or intrathecally.

26. (canceled)

27. The method of claim 19, wherein the AAV is administered at a dose of at least about 5×10⁹ vg.

28. The method of claim 19, wherein the administering of the rAAV does not elicit immune response.
